(19) United States
(12) Patent Application Publication
(10) Pub. No.: US 2024/0321420 A1
(43) Pub. Date: Sep. 26, 2024

(54) SYSTEMS AND METHODS FOR PROVIDING MEDICINE RECOMMENDATIONS

(71) Applicant: EQALIS GROUP NEW ZEALAND LIMITED, Hamilton (NZ)

(72) Inventors: Greg Charles MISSON, Hamilton (NZ); Elizabeth Anne PLANT, Hamilton (NZ); Shane RUTHERFURD, Hamilton (NZ)

(21) Appl. No.: 18/257,522

(22) PCT Filed: Dec. 14, 2021
(86) PCT No.: PCT/NZ2021/050221

§ 371 Date: Jun. 14, 2023

(30) Foreign Application Priority Data
Dec. 14, 2020 (NZ) 766629

(51) Int. Cl. G16H 20/10; G16H 10/60; G16H 70/40

(52) U.S. Cl. CPC G16H 20/10; G16H 10/60; G16H 70/40

(57) ABSTRACT

The technology relates to systems and methods for providing medicine recommendations using a population model generated from aggregate patient information. The systems and methods include a feedback system whereby the efficacy and side effects experienced by patients can be linked to their genetic information to enhance the accuracy of the population model.

### 1. CROSS-REFERENCE TO RELATED APPLICATIONS

The present application is a national phase application under 35 U.S.C. § 371 of International Application No. PCT/NZ2021/050221 filed Dec. 14, 2021, which claims the benefit of and priority to New Zealand Patent Application No. 766,629 filed Dec. 14, 2020, the contents of both of which are incorporated by reference in their entireties herein.

### 2. TECHNICAL FIELD

The present technology relates to systems and methods of providing medicine recommendations. The technology may find particular application in providing recommendations for prescriptions based on biological, pharmacogenetic and/or pharmacokinetic data. However, this should not be seen as limiting on the technology.

### 3. BACKGROUND

The purpose of a medicine is to aid in the treatment of a medical condition, or the symptoms of the medical condition. However, individual pharmacokinetics, therapeutic efficacy, potential side-effects and the severity of the side-effects vary across a population.

In the case of prescribed medicines, it is generally the prescribing physician who determines, based on the information available, which medicines an individual should be prescribed. Physicians often have access to information such as the individual's medical history, allergy information, age, weight and ethnicity. However, there are limitations to the information available to prescribing physicians in order to correlate the patient's clinical information to the likely occurrence of any known side-effect or to the likely efficacy of the medication.

Even if full clinical information is available to the prescribing physician it can be difficult to balance the various competing factors. This is particularly so where the disease to be treated by the medicine is an unusual one or an appropriate medicine is one with which they are not familiar.

In the case of unregistered, off-the-shelf, or non-prescription based medications, it is left to the customer to determine which medicine is suitable for the treatment of their medical condition. Unfortunately, generally little to no information is available to the public regarding the likely therapeutic efficacy or likelihood of any side-effects occurring. Furthermore, the information that is available is not tailored to the customer's biology.

Additionally, given the wide range of factors which can affect therapeutic efficacy and side-effects of a medication, it can be difficult to provide useful recommendations as to the best medication to treat any given medical condition. Further complicating matters is the interactions of various factors. For example, a medication may be suitable for use by an individual with an existing condition such as asthma, however when combined with other factors such as age, weight and ethnicity the likelihood of developing negative side-effects may increase significantly. In addition, where multiple medications are used they can interact with one another e.g. affect metabolism, therapeutic efficacy and negative side-effects. Requiring a doctor or prescribing physician to consider these factors can cause a paralysing effect where there is simply too much information to process. In addition, a large number of contraindications or potential drug interactions may be flagged which can cause “alert fatigue”, further affecting their ability to provide the best recommendations.

Recent medical developments such as genetic testing has allowed an additional range of patient specific factors to be identified which influence the therapeutic efficacy and risk of presenting with negative side-effects for a given medication(s). There is therefore a need to provide optimised medicine recommendations based on various biological, pharmacogenetic and pharmacokinetic information to improve the therapeutic efficacy of medicines, as well as reducing the likelihood or severity of any negative side-effects.

It is an object of the present invention to address one or more of the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### 4. BRIEF SUMMARY

According to one aspect of the technology there are provided systems and methods for providing medicine recommendations.

According to another aspect of the technology there are provided systems and methods for providing medicine recommendations based on at least one of biological, pharmacokinetic and pharmacogenetic data.

According to another aspect of the technology there are provided systems and methods for providing medicine recommendations, wherein the systems and methods comprise:
a list of formulations, said formulations comprising a first medicine containing a first active compound and a second medicine containing a second active compound,
wherein the formulations of the first medicine include a first formulation having a first concentration of the first active compound and a second formulation having a second concentration of the first active compound,
and further wherein the formulations of the second medicine include a third formulation having a first concentration of the second active compound and a fourth formulation having a second concentration of the second active compound, and
wherein the systems and method provide a medicine recommendation which contains both of:
one of the first formulation and the second formulation, and
one of the third formulation and the fourth formulation.

According to another aspect of the technology there are provided systems and methods for providing recommendations for a medicine to administer to a patient based on at least one of pharmacokinetic information relating to the patient and pharmacogenetic information relating to the patient.

According to a further aspect of the technology, there is provided a method of recommending a medicine to administer to a patient, the method comprising:
A) testing at least one genetic sample from the patient to determine the presence or absence of at least one single nucleotide polymorphism (SNP) associated with a risk factor for administration of the medicine;
B) testing at least one genetic sample to assess at least one SNP associated with the patient's likely metabolism rate of at least one active ingredient in the medicine;
C) providing an indication of the patient's suitability to be administered the medicine based on the assessed SNPs.

According to a further aspect of the technology, there is provided a system configured to provide a recommendation as to an appropriate medicine to administer to a patient, the system comprising:
an input system for receiving at least one piece of genetic information,
wherein the at least one piece of genetic information includes at least one single nucleotide polymorphism (SNP) associated with a risk factor for administration of a medicine, and
the patient's likely metabolism rate of at least one active ingredient in the medicine; and
a processing system that is configured to provide an output indicative of a medicine recommendation for the patient based on the patient information.

According to a further aspect of the technology, there is provided a system to generate prescriptions for administration of medicine to a patient, the system comprising:
an input system configured to receive patient information,
wherein the patient information comprises patient metabolism information and patient risk information,
a processing system configured to generate a medicine recommendation using the patient metabolism information in combination with the patient risk information, and
an output system configured to generate a prescription containing a recommendation for a medicine to administer to the patient.

According to a further aspect of the technology, there is provided a device configured to generate prescriptions for administration of medicines to a patient, the device comprising:
an input system configured to receive patient information,
wherein the patient information comprises patient metabolism information and patient risk information,
a processing system configured to generate a medicine recommendation using the patient information, and
an output system configured to generate a prescription containing the medicine recommendation based on the patient metabolism information and the patient risk information.

According to a further aspect of the technology there is provided a system to generate a recommendation for a medicine for administration to a patient, the system comprising:
an input system configured to receive patient information,
a processing system configured to generate a recommendation for the medicine using the patient information, and
an output system configured to generate a prescription containing the medicine recommendation based on the patient information.

According to a further aspect of the technology, there is provided a method of providing a recommendation for a medicine to administer to a patient, the method comprising:
A) receiving information regarding at least one of the patient's diagnosed medical conditions and one or more symptoms experienced by the patient;
B) receiving patient information comprising physiological and genetic information;
C) providing dosage recommendations based on the patient information.

According to a further aspect of the technology, there is provided a system for providing a dosage recommendation for a medicine to administer to a patient, the system comprising:
an input system configured to receive patient information comprising physiological and genetic information;
a processing system configured to generate a dosage recommendation for the medicine using at least one dosage factor and the patient information; and
an output system configured to provide an output representing the dosage recommendation.

According to a further aspect of the technology, there is provided a device for providing a dosage recommendation for a medicine for administration to a patient, the device comprising:
an input system configured to receive patient information comprising physiological and genetic information;
a processing system configured to generate a dosage recommendation for a medicine using at least one dosage factor and the patient information;
an output system configured to provide an output representing the dosage recommendation.

According to a further aspect of the technology, there is provided a system for providing a recommendation for a medicine for administration to one or more patients, the system comprising:
an input system configured to receive patient information from a plurality of patients, wherein the patient information comprises genetic information including information relating to at least one single nucleotide polymorphism,
a processing system, and
an output system configured to provide a medicine recommendation for a patient based on the patient information,
a feedback system configured to receive clinical feedback regarding symptom control;
wherein the processing system is configured to aggregate patient information from the plurality of patients to create a population model, and
further wherein the processing system is configured to modify the population model based on the feedback received by the feedback system to create an improved population model, and
further wherein the processing system is configured to provide medicine recommendations to one or more patients based on the improved population model.

According to a further aspect of the technology, there is provided a system for providing a recommendation for a medicine for administration to a patient, comprising:
an input system consisting of at least one electronic device configured to receive patient information,
a processing system comprising at least one processor,
a computer readable storage medium having computer readable program code embodied therewith and executable by the at least one processor,
the computer readable program code being configured to provide medicine recommendations based on the patient information and at least one dosage factor.

The present specification describes technology relating to medicines. One application for the technology is cannabinoid containing medicines and reference will be made herein as such.

However this should not be seen as limiting on the technology and other medicines as envisaged as being suitable for use with the present technology.

In various forms, the cannabinoids include CBD and THC, CBC, CBG, CBDV, THCV, THC delta 8, THC delta 9, cannabinoid acids (CBDA, THCA) varin analogues, endogenous counterparts and pharmaceutical equivalents e.g. synthetic cannabinoids. Reference herein will be made accordingly. It should be understood that the disclosure herein may be inclusive, e.g. the cannabinoids include analogues and other compounds having the same or substantially similar metabolic pathways and biochemical actions. For instance, to the extent that THCV has substantially the same biochemical action for treatment of a given medical condition as THC, then it will also be covered within the scope of the present technology. The same reasoning applies for CBD and CBDV, and to the extent that CBDV has substantially the same biochemical action for treatment of a given treatment of a condition as CBD then CBDV will also be considered within the scope of the present technology.

In addition, the present technology may find application with any medicine e.g. varin analogues, irrespective of whether the analogues have the same or substantially similar biochemical pathway to the compounds and single nucleotide polymorphisms (SNPs) described herein. In addition, the foregoing statements refer to systems, methods and devices which use one or more single nucleotide polymorphisms (SNPs) which are associated with metabolism or a risk factor. The inventors envisage that in some embodiments of the technology, the systems, devices and methods described herein may be implemented using a subset of, i.e. only one of, the described SNPs. For example, in relation to cannabinoid containing medicine recommendations, these recommendations may be provided based on THC metabolism and possible psychosis risk alone, without considering CBD metabolism or possible neurocognitive impairment risk. In alternative embodiments, any combination of SNPs may be used without departing from the scope of the present technology.

It should be further appreciated that reference to genetic information including at least one SNP should be understood to include information about the absence of the relevant SNPs.

In various embodiments, the patient metabolism information includes an assessment of at least one single nucleotide polymorphism (SNP) associated with metabolism of at least one active ingredient of a medicine. For example, in cannabinoid containing medicines, the SNP may be associated with the patient's metabolism rate of at least one of cannabinoid compound.

In embodiments of the technology comprising cannabinoid containing medicines, the patient metabolism information may include the patient's likely rate of metabolism of at least one of cannabidiol (CBD), CBDV and tetrahydrocannabinol (THC), THCV, THC delta 8, THC delta 9 or varin analogues thereof.

In various embodiments, the patient risk information may include an assessment of at least one single nucleotide polymorphism SNP associated with the patient's possible psychosis risk. For example, in cannabinoid containing medicines the possible psychosis risk may be due to administration of at least one of tetrahydrocannabinol THC, THCV, THC delta 8, THC delta 9 or varin analogues thereof.

In various embodiments, the patient risk information may include an assessment of at least one single nucleotide polymorphism SNP associated with the patient's neurocognitive impairment risk. For example, in cannabinoid containing medicines the neurocognitive impairment risk may be due to administration of at least one of tetrahydrocannabinol (THC), THCV, THC delta 8, THC delta 9 or varin analogues thereof.

In various embodiments, the patient risk information may include an assessment of at least one single nucleotide polymorphism (SNP) associated with the patient's potential addiction risk. For example, in cannabinoid containing medicines, the potential addiction risk may be due to administration of a compound e.g. a cannabinoid compound. The patient risk information and patient metabolism information, and single nucleotide polymorphism are discussed in more detail below.

In various embodiments, the present technology can use an assessment of single nucleotide polymorphism(s) to make decisions about patient medication recommendations. Reference herein will therefore be made to the assessment of a single nucleotide polymorphism(s) in specific genes.

This assessment preferably involves determining the presence or absence of a SNP in a specific gene(s)—which is/are a pharmacogenetic indication of a patient's likely response to administration of a compound or active ingredient e.g. a cannabinoid.

In various embodiments, the single nucleotide polymorphism(s) indicative of possible psychosis risk may be present in at least the patient's AKT1 gene. For example, the SNP may include any one or more of rs1130233 and rs2494732. Other SNPs associated with psychosis risk may be known to those skilled in the art. In other examples, the SNP indicative of possible psychosis risk may be present in the COMT gene.

In various embodiments, the single nucleotide polymorphism(s) indicative of metabolism rates, particularly for CBD and/or THC metabolism may be present in at least one of the patient's CYP2C9, CYP2C19, 2D6 and 3A4 genes. Preferably the SNPs indicative of the patient's likely rate of metabolism of CBD are present in the CYP2C19, 2D6 or 3A4 genes, and the SNPs indicative of the patient's likely rate of metabolism of THC are present in the CYP2C9 gene.

In various embodiments, the single nucleotide polymorphism(s) indicative of neurocognitive impairment may be present in at least one of the catechol-O-methyltransferase (COMT) genes.

For example the SNP may be rs4680 or any other SNP known to those skilled in the art. In an alternative embodiment, the recommendations described herein may be based on full or partial genome mapping, including determining the presence or absence of single nucleotide polymorphisms in genes other than the CYP2C9, CPY2C19, AKT1 and COMT genes.

In an alternative embodiment, the single nucleotide polymorphism(s) indicative of addiction risk may be present in any gene that provides an indication of addiction risk. For instance, the SNP may be present in at least the FAAH C385A gene.

In various embodiments, the genetic sample(s) may consist of a saliva or blood sample. However, this should not be seen as limiting on the technology, and other forms of genetic samples suitable for use with the present technology include blood, tissue, hair or amniotic fluid may be used.

In an alternative embodiment, genomic information regarding the presence and nature of any single nucleotide polymorphisms may be available and provided without requiring further testing.

In various embodiments the physiological information may comprise information relating to the patient's body and can include at least one of weight, age, renal function, hepatic function, and medical conditions. Alternatively, the physiological information may also include other information e.g. medication already being taken.

In various embodiments, the present technology may be configured to consider pathways for drug interactions to determine a dosage recommendation.

In a various embodiments, the dosage recommendations may consist of a starting dose and incremental step up to a maximum daily dose. In other examples, the dosage recommendation may consist of a set daily dose.

In various embodiments, the first concentration of the first active compound may be different to the second concentration of the first active compound. For example, the first formulation may have a higher concentration of the first active compound than the second concentration or vice versa.

Similarly the first concentration of the second active compound, may be different to the second concentration of the second active compound.

In various embodiments, the input system comprises an electronic device. For example, the electronic device may include a smartphone, tablet, laptop, desktop or personal computer.

In various embodiments, the input system may comprise at least one input device. For example, the input device may be a keyboard, touch-screen interface, touchpad or computer mouse.

In various embodiments, the processing system may comprise a local application. For example, the local application may include machine readable code configured to be executed on a processor in an electronic device.

In various embodiments, the processing system may further comprise at least one remote application configured to provide the medicine recommendations. For example, the remote application may be a cloud or web-based application.

In various embodiments, the recommendation may comprise information about a/the medicine to be prescribed to the patient. For example, in embodiments of the technology, where a single nucleotide polymorphism associated with psychosis risk is detected, the recommendation may comprise a daily dosage of less than or equal to 1 mg of cannabinoids identified as producing psychotropic effects such as tetrahydrocannabinol (THC) or tetrahydrocannabivarin (THCV).

In other examples of the technology, where a single nucleotide polymorphism associated with psychosis risk is detected, the candidate's risk of neurocognitive impairment may also be assessed, and the recommendation may comprise a non-zero daily dose of cannabinoids identified as producing psychotropic effects such as tetrahydrocannabinol (THC) or tetrahydrocannabivarin (THCV) or any formula containing other cannabinoids with psychotropic effects. The non-zero daily dose may be determined to be one which is likely to have a low-risk of causing neurocognitive impairment.

In other embodiments of the technology, when at least one single nucleotide polymorphism associated with possible psychosis risk is detected, the recommendation may comprise a cannabinoid containing composition containing substantially no cannabinoid having a psychotropic effect such as tetrahydrocannabinol (THC), cannabidol (CBN), THCV, THC delta 8, THC delta 9 or any formula containing other cannabinoids with psychotropic effects or varin analogues thereof.

In various embodiments, the dosage recommendation may be dependent on the patient's likely rate of metabolism of at least one active ingredient, such as a cannabinoid compound e.g. CBD and THC.

In various embodiments, the recommendation may comprise a prescription based on the indicated suitability for a cannabinoid containing medicine(s).

In various embodiments, the graphical user interface may be provided on an electronic display.

In various embodiments, the system may include at least one application programming interface (API) for receiving one or more pieces of information. For example, the API may be configured to allow the system to receive patient information including genetic information.

In various embodiments, the output system may comprise a display for presenting the recommendation.

In various embodiments, the output system may comprise a printer for providing a printed prescription or electronic prescription.

In various embodiments, the machine learning algorithm may comprise at least one neural network.

In various embodiments, the system may be configured to update the population model periodically, and the method may involve updating the population model periodically e.g. as additional patient records or population data is obtained. In a yet further example, the model may be updated as each recommendation is made.

In another embodiment, the system may be configured to output a lookup table of dosage factors which can be used by the systems and the methods described herein. For instance, the dosage factor table can be updated periodically, as new data is added, as recommendations are made, manually, or using any other frequency or technique known to those skilled in the art.

In various embodiments, the systems, devices and methods according to the present technology may be configured to create or collect patient records and/or population data. The patient records and/or population data may be used by the machine learning algorithms to improve the recommendations provided by the processing system. Alternatively, the patient records and/or population data may be used to provide an improved medicine recommendation to a specific patient based only on that patient's patient record.

In various embodiments, the dosage factors may include dosage factors for one or more of:
Age;
Sex;
Ethnicity;
Weight;
Body mass index;
Pregnancy, and breast feeding status;
Organ function (such as kidney, or liver function, particularly relating to liver disease);
History of unstable tachyarrhymia, and/or unstable coronary disease;
Currently used medications (including over the counter medicines);
Potential known medications that cause serious clinically significant drug interactions;
History of drug/medication use;
Allergies or adverse drug effects;
Drug concentrations in the patient's body;
Genotype information;
Disease state;
Medical history;
Isoenzyme pathways;
Diagnosis of schizophrenia, psychoses, bi polar or suicide ideation/attempts; and
Diagnosis of Cannabis Use Disorder.

### 5. BRIEF DESCRIPTION OF THE DRAWINGS

Further aspects of the present technology will become apparent from the ensuing description which is given by way of example only and with reference to the accompanying drawings in which:

FIG. 1 shows a block diagram overview of a system according to the present technology.

FIG. 2 shows exemplary input systems according to the present technology.

FIG. 3 shows a flow diagram for validating patient credentials and obtaining medical information in accordance with the present technology.

FIG. 4a shows a device according to the present technology comprising a local application.

FIG. 4b shows a device configured to communicate with a remote application.

FIG. 4c shows a device comprising a local application configured to communicate with a remote application.

FIG. 5 shows a block diagram illustrating processing information structures according to the present technology.

FIG. 6 shows an exemplary user interface for a feedback system according to the present technology.

FIG. 7a shows an exemplary user interface for entering patient information in accordance with the present technology.

FIG. 7b shows a further exemplary user interface in accordance with FIG. 7a.

FIG. 7c shows a further exemplary user interface in accordance with FIG. 7a.

FIG. 7d shows a further exemplary user interface in accordance with FIG. 7a.

FIG. 7e shows a further exemplary user interface in accordance with FIG. 7a.

FIG. 8 shows an exemplary neural network according to the present technology.

FIG. 9 shows a flow diagram for determining relative THC and CBD concentrations based on analysing single nucleotide polymorphisms in accordance with the present technology.

### 6. DETAILED DESCRIPTION

### 6.1. Overview of an Embodiment of the Technology

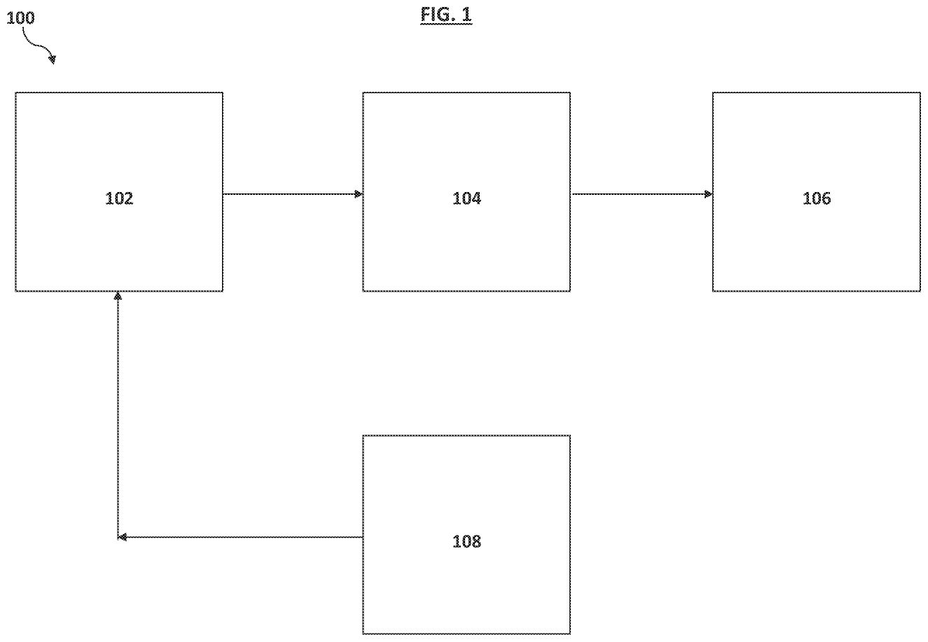

FIG. 1 is a flow chart which provides a high-level overview of a first embodiment of a system 100 for providing medicine recommendations according to the present technology. In general terms, the system 100 comprises an input system 102 configured to receive clinical information about a patient (“patient information”). For instance, the clinical information may include:
Age;
Sex;
Ethnicity;
Weight;
Body mass index;
Pregnancy, and breast feeding status;
Organ function (such as kidney, or liver function-particularily liver disease)
History of unstable tachyarrhymia, and/or unstable coronary disease);
Currently used medications (including over the counter medicines);
Potential known medications that cause serious clinically significant drug interactions
Allergies or adverse drug effects;
Drug concentrations in the patient's body;
Genotype information;
Disease state;
Medical history e.g. one or more of diagnosis of schizophrenia, pyschoses, bipolar, suicide ideation or attempts, and diagnosis of Cannabis Use Disorder;
Isoenzyme pathways; and/or
One or more genetic markers indicating the patient likely response to a medicine e.g. one or more specific pharmacogenetic genotypes.

The input system 102 is configured to provide the patient information to a processing system 104. The processing system 104 generates medicine recommendations based at least partially on the patient information. Various processing systems 104 are described herein, and it should be appreciated that any one or more of the processing systems 104 described may be used in the examples provided.

The recommendation(s) generated by the processing system 104 are then provided to an output system 106. In general terms, the output system 106 is configured to output the recommendation(s) for medicines to be prescribed to the patient(s) to which the patient information relates.

One application for the present technology is to assist a physician in providing medicine recommendations to a patient. For example, the present technology could be used to provide a recommendation for a medicine which is likely to have positive therapeutic benefits, with a low-chance of negative side effects. In this application, the output system 106 may be configured to provide the recommendation(s) to the physician who can review the recommendations and make a final decision of prescribing the medicine(s) to the patient. For example, the output system 106 may be configured to do at least one of the following: present the recommendations on an electronic device (such as a display), print the recommendations via a printer, send the recommendations to the physicians practise management system (PMS), or automatically generate a prescription for the medication (for example by interfacing with an electronic prescription system (EPS)).

Another application for the technology is to provide recommendations directly to the patient. For example, the output system 106 may provide the recommendation(s) directly to the patient via an electronic device such as a display, via an email, or as printed information via a printer.

In examples of the technology, the system 100 may further comprise a feedback system 108. The feedback system 108 is configured to enable data from one or more sources to be fed back into the input system 102. For example, the feedback system 108 may receive feedback from the patient.

This feedback may consist of subjective feedback such as feedback from the patient as to the therapeutic response or perceived efficacy of the medication due to their symptom control, or the presence and severity of any side-effects experienced. It is possible that the feedback may be provided using a known validated tool for assessing therapeutic effect as should be known to one skilled in the art e.g. available at https://www.practicalpainmanagement.com/sites/default/files/pain_scales_table.pdf. In some examples, the feedback may also comprise objective feedback such as clinical information on drug concentrations in the patient's body. It is also envisaged that the feedback may be provided by a clinician assessing efficacy of the medication.

For example, after a period of treatment (such as one month), the patient may be tested to determine the drug concentrations present in the patient's system. For example, the patient's blood plasma may be tested to determine drug concentrations. For example, for medicines comprising CBD or THC, the patient's blood may be tested to determine CBD and or THC concentrations in blood plasma. The patient's information can then be updated in the input system 102 and new recommendations generated by the processing system 104. For example, if testing shows a high drug concentration (such as CBD or THC concentration) in blood plasma (considering peak and trough levels), the processing system 104 may generate a medicine recommendation with a lower dose of the active ingredient (such as CBD or THC), or alternatively a lower dose of the same medication previously recommended.

One advantage of the present technology is that the feedback information may be linked to the clinical information provided to the input system 102 and the recommended medication. This allows the processing system 104 to create a population model linking clinical information feedback such as therapeutic efficacy, side effects experienced, metabolism rates (i.e. what drug concentrations remain in the patient's system after use) based on patient compliance with a prescription recommendation. therefore, the medicine recommendations provided by the present technology may be improved over time. Yet a further advantage of the present technology is that it may assist development of formulations to better treat medical conditions or adapt to meet patient specific clinical requirements, or assist in meeting regulatory requirements by providing proof of efficacy. These aspects and advantages of the present technology should become clearer from the following discussion.

### 6.2. Input Systems

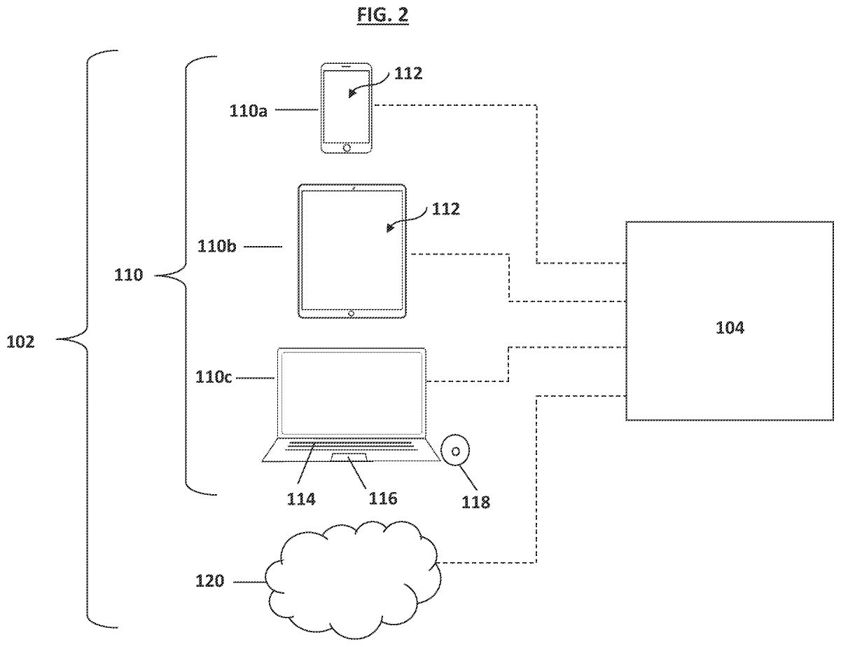

Referring now to FIG. 2 which shows a representative input system 102 with multiple input devices configured to provide patient information and/or feedback information to the processing system 104.

In examples, the input system 102 is provided with one or more electronic devices such as smartphones 110a, tablets or laptops 110b, and desktop or personal computers 110c. For example, data can be input to an electronic device 110 using a touch-screen interface 112, keyboard 114, or pointing device 116 (such as a mouse or joystick).

In examples of the technology, the input systems 102 may also be used to enter the feedback information to the feedback system 108. For example, a survey, questionnaire, or internationally recognised measurement tool (such as quality of life or pain scoring) may be presented to a patient via an electronic device 110a, b and/or c. It should be appreciated that the electronic device 110a, b and/or c which is used to enter the feedback information may be different to the electronic device 110a, b and/or c which was used to input the clinical information about the patient. For example, clinical information may be entered into an electronic device 110 at a physician's office. Following a period of treatment, the patient may be emailed a feedback form which they can complete remotely, such as on their personal electronic devices 110a, b and/or c at home.

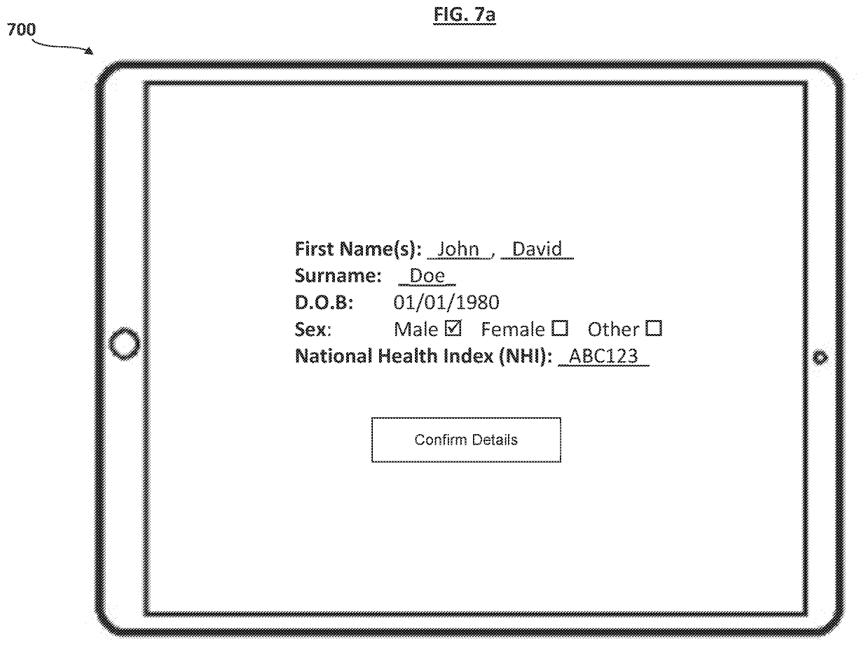

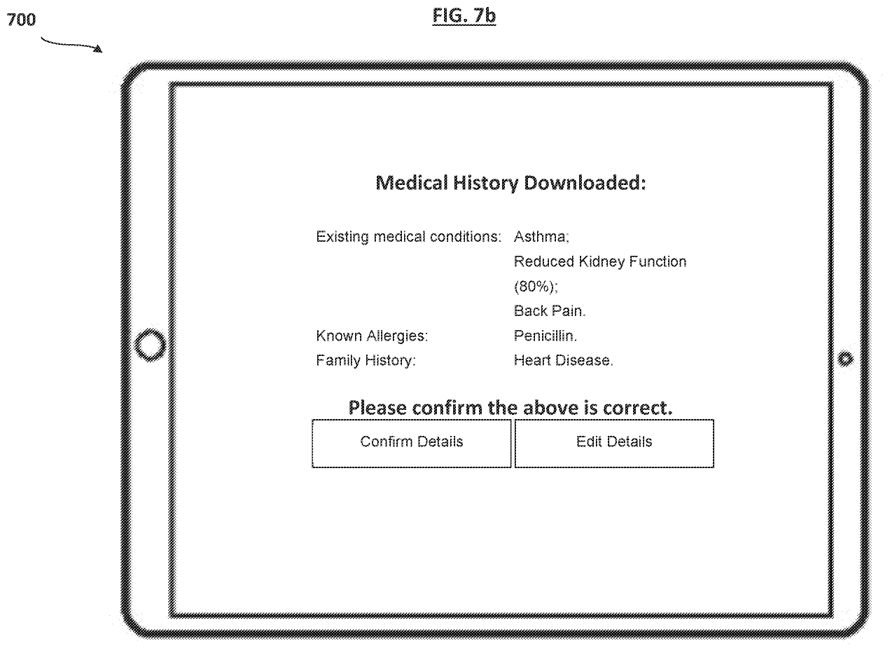

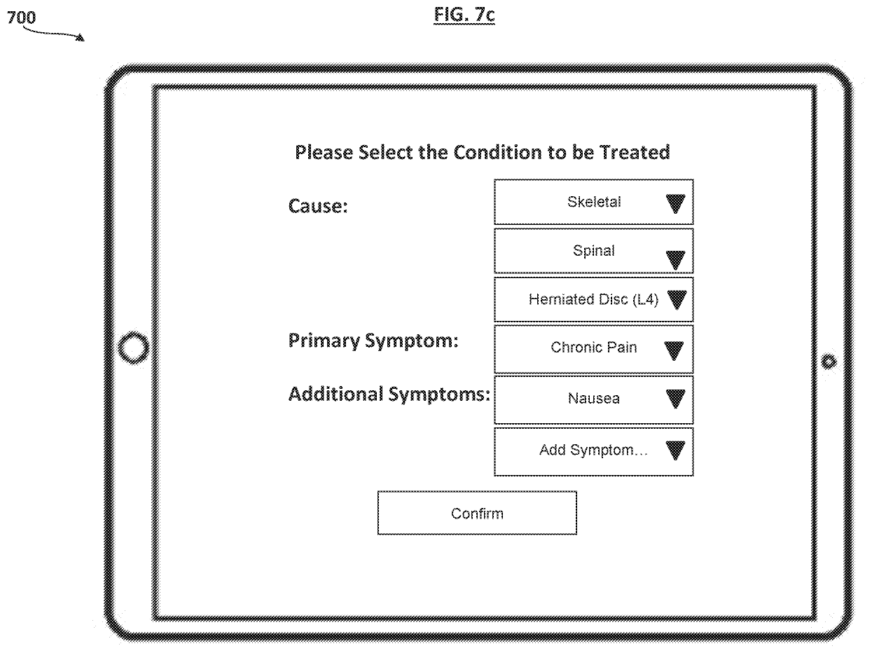

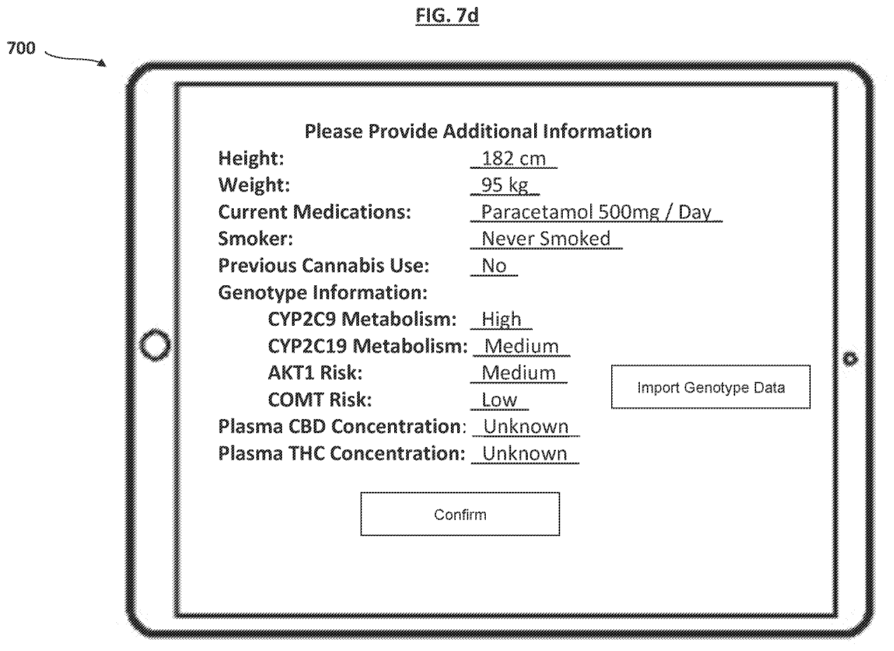

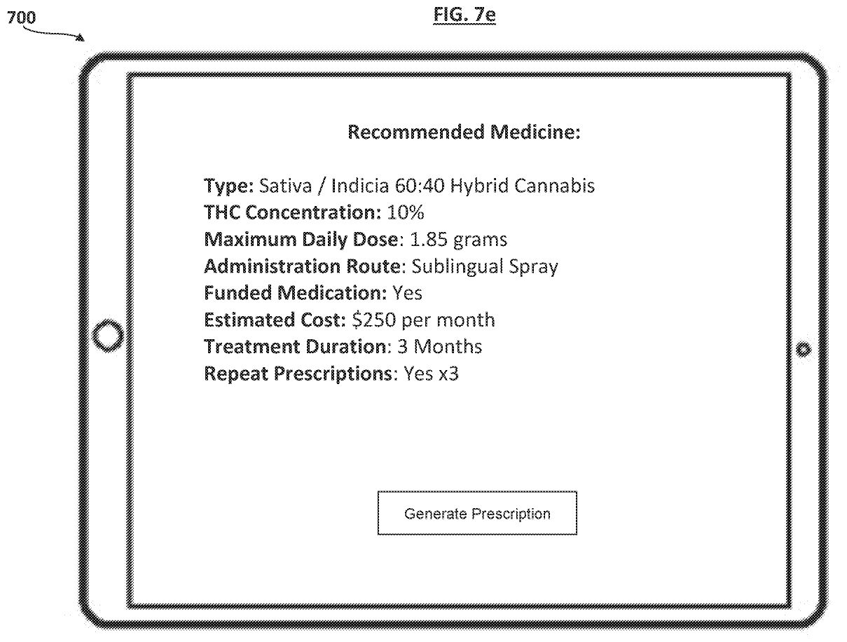

The input systems 102 described herein may comprise a User Interface (UI) configured to prompt the patient to enter specific information. Examples of a suitable UI is illustrated in FIGS. 7a to 7e which are discussed in detail below.

The input system 102 is configured to receive clinical information which relates to the person seeking medicine recommendations. This clinical information may include medical history, biological, pharmacokinetic and/or pharmacogenetic information as described herein. In examples, the input system 102 may also be configured to receive information about the symptoms which the person is looking to treat. For example, the symptoms may include pain, anxiety, and depression. In examples the input system 102 may also be configured to receive information relating to the cause of the condition. For example, the information may contain information about a dislocated joint, hernia, or broken limb.

It should be appreciated that references herein to entering patient data may include someone acting on the patient's behalf. For example, a doctor, physician, nurse, or caregiver may in some examples enter the clinical information and/or feedback on the patient's behalf.

In examples, the electronic devices 110 may be configured to connect to a removable storage 118 device such as a USB mass storage device or compact disk (CD) in order to retrieve patient information. For example, genome sequence information or medical history information may be provided on a removable storage device 118. Other examples of retrieving patient information include via wired or wireless transmissions, such as ethernet, WiFi, Bluetooth or NFC.

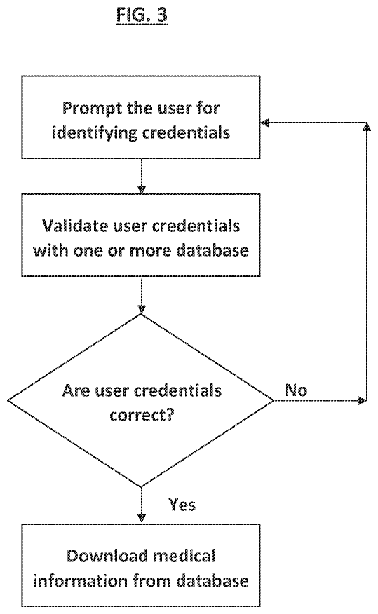

The input system may also be configured to receive information from a network or cloud-based data storage 120. One example of a method for obtaining medical information is illustrated in FIG. 3. Firstly, the patient is prompted to provide identifying credentials. This could include a unique identifier, such as a National Health Index (NHI) number, an Accident Compensation Corporation (ACC) claim number, an individual healthcare identifier (IHI), Medicare number, or any other form of unique identifier. In examples, the patient is also prompted to provide their name, and date of birth, in order to confirm that the unique identifier is for the correct person.

These identifying credentials are then validated against one or more databases such as a patient information database or practice management database. Providing that the credentials are valid, the system may obtain medical history about the patient including medical history, biological, pharmacokinetic and/or pharmacogenetic information.

In other examples, the system 100 may comprise its own database of patient information, and the patient may provide sufficient identifying credentials, such as log-in information in order to access any previously saved information.

### 6.3. Processing Systems

In embodiments, the processing system 104 is configured to provide recommendations on the best medicine to prescribe a patient, based at least partially on the information provided by the input system 102.

Other sources of information which may be used to determine suitable medication regimen recommendations include one or more of the following:
Medical research published in journals or articles which suggest the use of certain medications to treat particular conditions or symptoms;
Medicine certification documentation from regulatory agencies;
Dosing factors which can be used to determine whether the patient should be recommended a higher or lower dose of medication based on the information provided by the input system 102;
Feedback from other patients e.g. subjective feedback on therapeutic efficacy and severity of side-effects; and
Cost and funding status of various medications.

In one example the processing system 104 comprises at least one processor, and a computer readable storage medium having computer readable program code which is executable by the at least one processor, the computer readable program code being configured to provide medicine recommendations as described herein.

### 6.3.1. Application Architecture

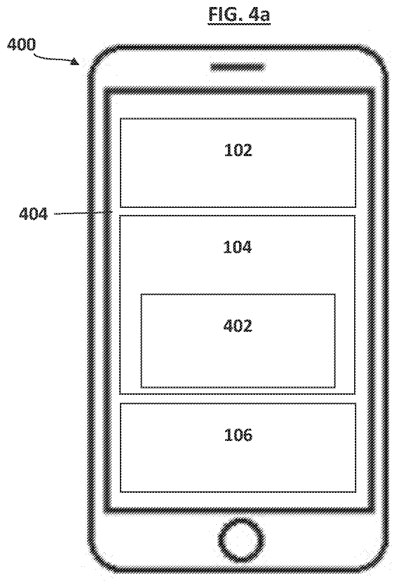

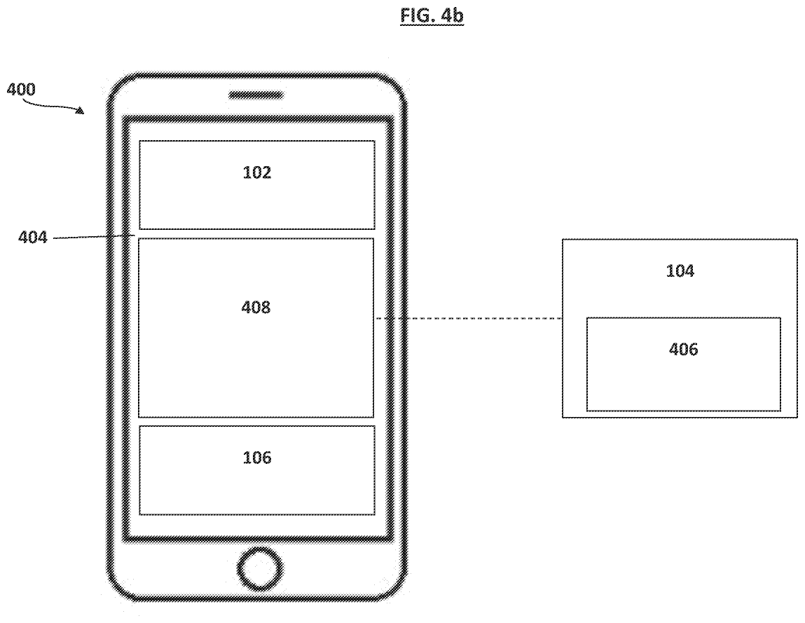

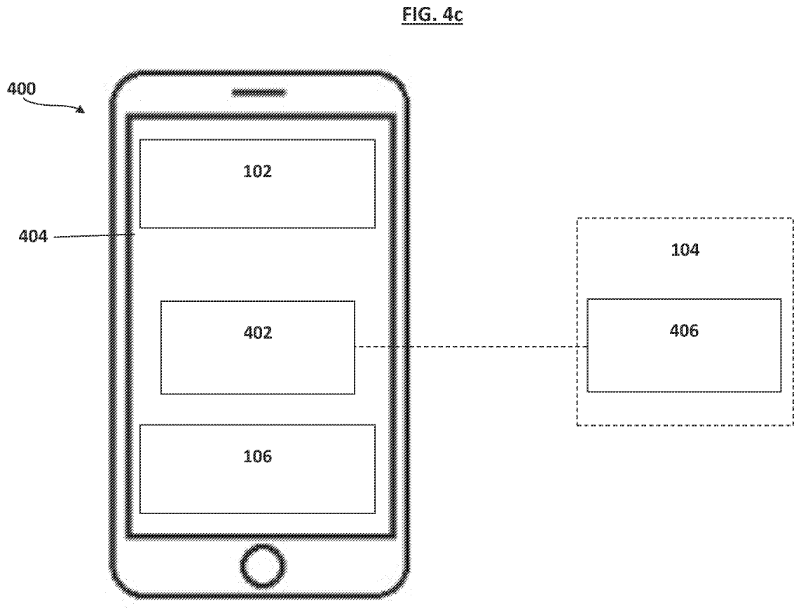

Referring now to FIG. 4a which shows an example of the technology in which an electronic device 400 is configured to facilitate use of the technology by a patient. The electronic device illustrated is a smartphone, however this should not be seen as limiting on the technology.

As shown, the electronic device 400 comprises an input system 102 configured to receive patient information. In use this would be provided by a touch screen interface or a connected peripheral such as a keyboard or mouse. The patient information is provided to the processing system 104, which in this example comprises a local application 402 installed on the electronic device 400.

The local application 402 includes machine readable instructions which are configured to be executed on a processing unit (such as a Central Processing Unit (CPU) or Graphics Processing Unit (GPU)) within the electronic device 400. In this example, the local application 402 processes the information provided by the input system 102 in order to provide medicine recommendations to the output system 106. The output system 106 in this example is the display 404 of the electronic device. However, this should not be seen as limiting on the technology, and the recommendations may instead be sent to another device such as a printer, or a web server.

In examples where local applications 402 are used it may be beneficial for the local application 402 to communicate the specific recommendations, and feedback to a remote server in order to allow for improvement of the processing system 104 recommendations over time. For example, the electronic device 400 may be configured to communicate the recommendations via a wireless network connection such as cellular data or WiFi. Where cellular data or WiFi networks are unavailable, the electronic device 400 may be configured to store this information and transmit it once a suitable network connection has been re-established.

One advantage of using local applications 402 is the ability to provide medicine recommendations in areas having poor network coverage, such as in disaster relief regions.

In another example of the technology illustrated in FIG. 4b, the electronic device 400 includes the same input system 102 and output systems 106 as previously described. However, the processing system 104 comprises a remote application 406 which is external to the electronic device 400. For example, the remote application 406 may be a web or cloud-based application. In this example, the electronic device 400 is configured to communicate with the remote application using a web-browser 408 on the electronic device 400.

One advantage of using a remote application 406 is the ability to utilise remote processing power.

Remotely processing the information can allow for faster results (particularly on slower devices) and reduced power consumption (particularly on battery powered devices).

In a yet further example of the technology illustrated in FIG. 4c, the processing system 104 comprises a combination of local application(s) 402 and remote application(s) 406. For example, a local application 402 may facilitate the entry of some clinical information, and in some examples perform some processing of the information provided. For example, the local application 402 may calculate a Body Mass Index (BMI) based on height and weight data provided by the input system 102. The local application 402 may also provide a secure means for transmitting the patient information to the remote application 406. For example, the local application 402 may encrypt the information communicated to the remote application 406.

The local application 402 may be configured to communicate with the remote application 406 via one or more Application Programming Interfaces (API), a web browser, or using a file transfer methods such as a File Transfer Protocol (FTP) or SSH File Transfer Protocol (SFTP). In should be appreciated that the foregoing communication methods are provided by way of example only, and those skilled in the art will be aware of other suitable communication methods that may be used.

### 6.3.2. Information Structure

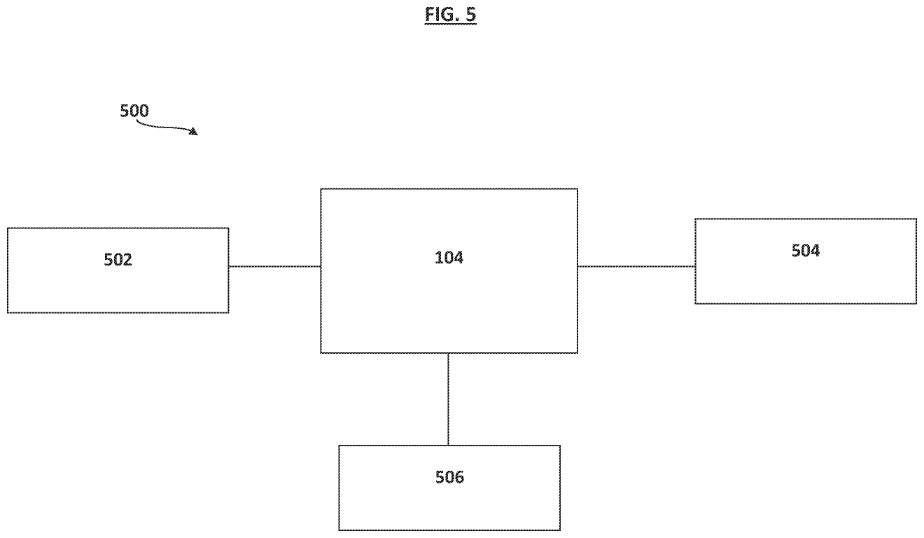

In general terms, the processing system 104 includes one or more of the following sets of processing information 500 as illustrated in FIG. 5:
Medication information 502;
Price and Availability Information 504; Dosage Factors 506; and
Medication coding data such as ULM (Universal medicines List) coding or equivalent SNOWMED compatible coding.

In some applications of the technology, price and availability information 504 may not be available or used. For example, recommendations may be provided based on the best available medicine, irrespective of the price or availability of the medication. Accordingly, in some examples of the technology, the processing system 104 may provide a plurality of recommendations, and the patient or physician may select the appropriate medication based on the availability and/or price of the medication in the patient's local area.

The medication information 502 may include:
The concentrations of active ingredients in the medication. For example, for Cannabis-based medications this may include the tetrahydrocannabinol (THC) and Cannabidiol (CBD) concentrations.
The therapeutic effects of the medication. For example, the medicine may be intended for treatment of chronic pain, seizures, spasticity, nausea, headaches or inflammation.
The known side-effects of the medication. For example, confusion, dizziness, dry mouth, stomach cramps etc.
Recommended dosages for the medication. For example, these may be provided at different levels for adults or children, or at different levels based on weight.
Forms the medication is available in. For example, as a spray, tablet, oil, or as organic material.
Any contraindications. For example, a list of medicines which should not be used in combination with the medication, or a subset of the population for whom the medication is not suitable i.e. those who are pregnant/breastfeeding, history of psychosis or have a serious heart condition, or liver disease.

The pricing and availability information 504 may include:
A unit cost for the medication.
Whether the medication is funded, and if so the conditions for the funding. For example, whether the medication is free for those aged 65 or older.
Informed patient consent if the medicine is unapproved or used off license.
Information as to the availability of the medications. For example, whether the medication requires a prescription, or is controlled by specific regulations (such as the misuse of drugs act 1975).

The dosage factor information 506 may include information relevant to the recommended dosages and concentrations of active ingredients recommended. For example, the dosage factor information 506 may include:
Age factors. For example, containing preparations containing only CBD may be recommended for females under the age of 20 and for males under the age of 25.
Sex factors. For example, a lower dose may be recommended for men or women.
Weight factors. For example, a higher dose may be required for a heavier person.
Organ function. For example, those with reduced kidney or liver function may require a lower dose.
Factors relating to other medications. For example, a reduced or increased dosage may be required if the patient is currently a medication with a similar active ingredient. In addition, there are known interactions between different drugs which need to be considered (such as CBD reducing the metabolism rate of drugs such as clobazam resulting in high levels, and THC and CBD increasing the levels of warfarin).
Disease state factors. These can impact the decision whether or not to use CBD or THC products, and may impact the dosages accordingly. Some disease states are contraindicated such as schizophrenia.
Factors relating to past medication/substance use. For example, past Cannabis use may develop resistance to the effects of CBD and/or THC and if someone has taken THC previously they may be started on a higher initial dose.
Factors based on the current medicine concentrations in a patient's body. For example, high levels of CBD in the user's body may indicate that the patient should be recommended a medication comprising a lower dose of CBD.
Medical history factors. For example, certain dosages may need to be reduced if there is a history of psychosis or other health conditions with the individual or in their immediate family.
Genotype factors. For example, whether their genetic profile indicates that they may have a possible psychosis or impairment risk from THC containing medications or that due to slow metabolism rates they may need a dose reduction.
Isoenzyme pathway factors. For example, to reduce the chances of adverse drug interactions.
Pharmacodynamic factors that may impact on drug levels such as the concomitant administration of Central Nervous System medications
An incremental dosing regimen—This may be comprised of a starting dose and incremental step up to a maximum daily dose.

It should also be understood that the system may enable a prescribing physician to override a recommendation due to clinical needs. This may be particularly useful in situations where a cost benefit assessment or clinical risk assessment suggests that the risk of potential adverse side affects are outweighed by the benefits e.g. in prescribing medicines containing THC for pain relief in males under 25 years and females under 20 years, or in treating autism and behavioural issues in children.

### 6.3.3. Updating the Processing System

One advantage of the systems and methods described herein is the ability for the processing information 500 to be easily updated and maintained. For example, as new medicines are approved, the processing information 500 relating to these medicines can be added to the processing system 104 to improve the recommendations provided. In other examples, the processing system 104 may be updated based on feedback from the patient.

In examples of the technology, where local applications 402 are used, it is desirable for a master copy of the processing information 500 to be maintained. A copy of this processing information 500 can then be bundled into application updates, or alternatively updated during runtime of the local application. In other examples of the technology, where remote application(s) 406 are used, the remote application 406 may contain the master copy of the processing information 500.

In examples of the technology, the processing information 500 can be centrally managed. For example, a person or team of people may be tasked with manually updating the processing information 500 described herein as new information is made available.

In another example, the processing information 500 may be automatically updated as information becomes available. For example, medication information 502 may be extracted from one or more publicly available registers (such as a universal list of medicines) using techniques known in the art such as API's, importing data structures such as database files, or by scraping content from one or more websites.

Similar techniques may be employed to update information relating to the price and availability information 504 as well as publicly known or published dosage factor information 506.

In examples discussed below, the dosage factor information 506 may be updated based on feedback provided by the feedback system 108. Accordingly, the dosage factor information 506 of the present technology may be more accurate than existing systems as it may be tailored to specific patient information such as medical history, biological, pharmacokinetic and/or pharmacogenetic information.

In examples, the factors relating to other medicines may be obtained by interfacing with an external reference resource such as “Stockley's Drug Interactions”, British National Formulary (BNF), or New Zealand National Formulary (NZF) via means known to those in the art such as using one or more APIs.

In yet further examples, the processing information 500 may be updated by an artificial intelligence system as described herein. For example, the medication information 502, price and availability information 504 and/or the dosage factor information 506 may be updated using an artificial intelligence system such as machine learning.

### 6.3.4. Patient Information

### 6.3.4.1. Patient Records

In examples of the technology, the systems and methods described herein may be configured to generate, collect and/or store patient records. The patient records may contain one or more of the following:
Identifying information about the patient;
The clinical information provided by the patient;
Details of medication recommended by the processing system 104;
Any feedback provided by the patient; and
Any other information the patient has authorised.

In some examples of the technology, the patient records may be linked to specific patient log-in credentials, so that a patient can access their patient record at a later stage. For example, when providing feedback to the feedback system 108. In other examples, the patient may be automatically linked to their patient record when providing feedback information. For example, a patient may be asked to follow a link or URL to access a survey or questionnaire. The link or URL may contain sufficient identifying information to link the patient's feedback to their patient record.

In other examples, each time the patient interacts with the systems described herein, a new patient record is generated.

In some examples of the technology, it may be beneficial for the systems described herein to communicate with an external database, such as a practise management system (PMS). In this way the patient record (or a subset of information from the patient record) may be shared with the PMS in order to ensure that the PMS system is up-to-date. Methods of communicating information between databases should be well known to those skilled in the art. For example using API's, web interfaces, importing files, or using file transfer methods.

### 6.3.4.2. Population Data

In some examples of the technology, the input systems 102 and/or feedback systems 108 may be configured to collect population data. This population data may be used to create a model of the population in order to provide improved recommendations in the future.

In some examples this population data may be anonymised so as to ensure the privacy of the patient's information. The population data may be collected in conjunction with the patient records described herein, as an alternative to the patient records, or may be generated from the patient records.

In some examples, the anonymised population data includes one or more of the following:
Clinical information about the patient;
A record of medications recommended to the patient;
Feedback as to the side-effects and therapeutic efficacy of the medication; and
Specific formulation compositions such as ratio of CBD and THC and whether full spectrum or pure isolates

### 6.4. Output Systems

Various output systems may be used in accordance with the present technology. For example, where electronic devices 110 are used, the output system may include an electronic display, or printer. In yet further examples, the output system 106 may be configured to generate a file, such as an email or text document.

In further examples of the technology, the output system 106 may be configured to generate a prescription for one or more of the recommended medications. For example, an electronic or paper-based prescription may be generated. The prescription may be automatically generated as the recommendation is generated. In other examples the prescription may require input by a prescribing physician.

In yet further examples of the technology, the output system 106 may be configured to interface with a medical centre's practice management software (PMS). For example, the recommendation may be transferred to a PMS so that the medical centre has an automated record of the recommendation, as well as details of the prescription information (if applicable). The prescription may be generated by the New Zealand electronic prescription service through an API to the dosing system.

### 6.5. Feedback Systems

One advantage of the present technology is the ability to update the processing system 104 to improve recommendations over time. One method of achieving this is using the feedback system 108.

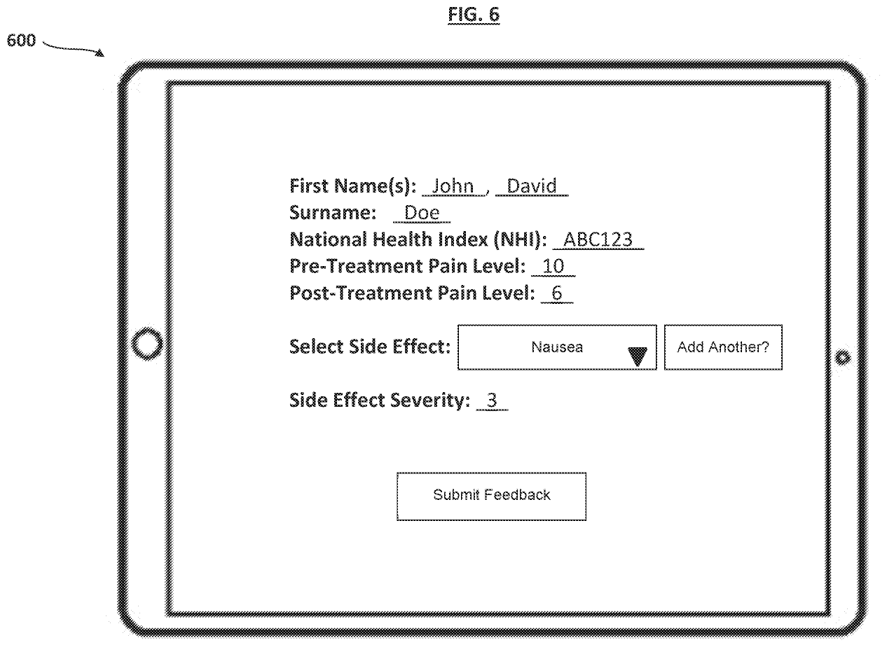

In one example of the technology, the feedback system 108 comprises questionnaire or survey-based feedback 600 as shown in FIG. 6. In this example, a patient is prompted to provide identifying credentials, such as their name and a unique identifier e.g. a national health index number. These credentials are used to link the feedback to the patient information. In some examples, the feedback system 108 may automatically link the feedback to the medications that the patient is taking. In other examples, the patient may be prompted to enter information about the medication they are taking, and the dosages thereof. This may be particularly beneficial if the patient is taking a higher or lower dose than originally prescribed.

The patient is then prompted to provide feedback as to the therapeutic efficacy of the medication. In the illustrated example, the medication was prescribed for treatment of pain, and accordingly the patient is prompted to provide feedback as to how well managed the pain systems are post-treatment. In, examples, the feedback system may populate a field which shows the patient how they rated their symptoms (pain in this case) prior to starting the treatment (for example using a validated pain score measurement tool). The patient can then provide direct feedback as to whether the prescribed treatment has been effective, and quantify how effective the treatment has been.

The patient is also prompted to provide feedback as to whether they have experienced any side-effects while taking the medication, and if so, the severity of the side effects.

The feedback is then provided to the processing system 104 which records the feedback against the specific patient's clinical information, for example the feedback may be used to update a patient record as described herein. In this way, if another patient presents with similar patient information in the future, the processing system 104 can then tailor the recommendation based on whether the treatment was successful for similar candidates in the past.

It should be appreciated that while the survey-based feedback system illustrated in FIG. 6 is shown on an electronic device, this should in no way been seen as limiting on the technology, and in other examples, the feedback may be obtained via a phone call, or by completing a paper questionnaire.

### 6.6. Examples

### 6.6.1. Cannabinoid Containing Medicines

One advantage of the present technology is the ability to provide an improved medicine recommendation system for medicines containing cannabinoids. Note that these medicines may be referred to as Cannabis-based medicines.

When it comes to medicines comprising cannabidiol, there are specific tests which can be used to provide additional information to a feedback system 108 in accordance with the present technology. For example, tests are available for determining concentrations of CBD and THC within a patient's body, such as blood plasma testing. Accordingly, dosage recommendations can be optimised by using pharmacogenetic information, and these recommendations can be optimised by subsequently testing for latent drug concentrations post-treatment.

### 6.6.1.1. Single Nucleotide Polymorphisms

The inventors have surprisingly found that single nucleotide polymorphisms (SNPs) can be used to determine appropriate medicine prescriptions for a patient. While the present specification describes embodiments of the technology with reference to SNPs associated with metabolism, neurocognitive impairment, and psychosis risk for cannabinoid containing medicines, this should not be seen as limiting on the technology. For example, the presence of SNPs in the VKORC1, CYP3A4, CYP1A2 and CYP2C9 genes are known to affect the patient's response to the anticoagulant drug warfarin. Accordingly, the technologies described herein may be applied to any combinations of genetic and clinical information in order to provide improved medicine recommendations.

When it comes to cannabinoid containing medicines, specific SNPs can be used to provide recommendations based on the patient's likely rate of metabolism of THC and CBD. In addition, specific SNPs can be used to determine likely risk factors for psychosis and neurocognitive impairment which can improve medicine prescriptions, by reducing the likelihood or severity of related negative side-effects. For example, single nucleotide polymorphisms in the:
CYP2C9 gene can be used to predict a patient's likely rate of metabolism of THC;
CYP2C19 gene can be used to predict a patient's likely rate of metabolism of CBD;
AKT1 gene can be used to predict a patient's likely psychosis risk as a result of consuming THC; and
COMT gene can be used to predict a patient's like chance of neurocognitive impairment as a result of consuming THC.

For example, any one or more of the following CYP2C19 SNPs may have a negative effect on cannabinoid metabolism: rs4244285, rs4986893, rs28399504, rs56337013, rs72552267, rs72558186, rs41291556, rs17884712, rs6413438, rs192154563, rs140278421, rs118203757, rs118203759, and rs12769205. Conversely, rs12248560 may have a positive effect on cannabinoid metabolism.

Any one or more of the following CYP2C9 SNPs may have a negative effect on cannabinoid metabolism: rs1799853, rs1057910, s56165452, rs28371686, rs9332131, rs7900194, rs28371685, rs9332239, rs72558187, rs72558190, rs72558188, and rs57505750.

Any one or more of the following CYP3A4 SNPs may have a negative effect on cannabinoid metabolism: rs55785340, rs72552799, rs67784355, rs12721629, rs4986909, rs12721627, rs4987161, rs67666821, rs35599367, and rs138105638.

CYP1A2 is known to affect metabolism of certain drugs. For example individuals who carry one or more CYP1A2*1C alleles are “slow” caffeine metabolizers, whereas carriers of the variant CYP1A2*1F are “fast” caffeine metabolizers.

CYP2D6 is understood to be related to the metabolism of a large number of drugs including dextromethorphan.

Other examples include:

| Single Nucleotide | | |
|---|---|---|
| Polymorphisms (SNPs) | Gene | Risk Factor |
| rs1535255. | CNR1 (Cannabinoid | Modest association with alcohol dependence. |
| | Receptor 1). | |
| rs1535255 | CNR1 (Cannabinoid | Impulsivity in Southwest California Indians. |
| rs2023239, | Receptor 1). | |
| rs1049353, and | | |
| rs806368. | | |
| rs6454674 and | CNR1 (Cannabinoid | Substance dependence. |
| rs806368. | Receptor 1). | |
| rs1049353. | CNR1 (Cannabinoid | A pharmacogenetic factor for antipsychotics. |
| | Receptor 1). | Visceral and intermuscular fat mass. |
| | | Plasma cholesterol levels. |
| | | Association with ulcerative colitis |
| | | susceptibility and the Crohn's disease |
| | | phenotype. |
| | | Predisposition to weight gain in Europeans. |
| rs6454674, and | CNR1 (Cannabinoid | Increased risk for cocaine dependence in the |
| rs806368. | Receptor 1). | European-American sample. |
| rs6454674, and | CNR1 (Cannabinoid | Risk for obesity. |
| rs10485170. | Receptor 1). | |
| rs2023239. | CNR1 (Cannabinoid | Substance abuse, functional changes in |
| | Receptor 1). | cannabinoid regulation, and cannabis |
| | | withdrawal. |
| | | Obesity. |
| | | Significant association with glutamate |
| | | pyruvate transaminase. |
| | | Reduced fronto-temporal white matter |
| | | volumes in schizophrenia patients as a result |
| | | of cannabis misuse. |
| | | Lifetime major depressive disorder and |
| | | suicidal behaviour in a population of opiate- |
| | | dependent outpatients. |
| | | Nicotine dependence, risk factor for Gilles |
| | | de la Tourette syndrome in Polish |
| | | population. |
| rs324420. | FAAH (Fatty Acid | Substance abuse, functional changes in |
| | Amide Hydrolase). | cannabinoid regulation, and cannabis |
| | | craving. |
| | | Association with weight gain after |
| | | antipsychotic exposure. |
| | | Obesity. |
| | | Risk factor for myocardial infarction, and |
| | | postoperative nausea, vomiting, alcohol |
| | | binge drinking tendencies, and respiratory |
| | | depression. |
| rs2501432, and | CNR2 (Cannabinoid | Increased risk of schizophrenia. |
| rs12744386. | Receptor 2). | |
| rs7766029. | CNR1 (Cannabinoid | Risk factor for depression. |
| | Receptor 1). | |
| rs1049353 and | CNR1 (Cannabinoid | Anorexia nervosa and bulimia nervosa. |
| rs324420. | Receptor 1) and | |
| | FAAH (Fatty Acid | |
| | Amide Hydrolase). | |
| rs2023239, and | CNR1 (Cannabinoid | Response to marijuana cues. |
| rs324420. | Receptor 1) and | |
| | FAAH (Fatty Acid | |
| | Amide Hydrolase). | |
| rs806368, and | CNR1 (Cannabinoid | Cannabis dependence. |
| rs806380 | Receptor 1). | |
| rs1049343, | CNR1 (Cannabinoid | Susceptibility marker for cutaneous T-cell |
| rs12720071, and | Receptor 1). | lymphoma. |
| rs806368. | | |
| rs806368. | CNR1 (Cannabinoid | Obesity and serum leptin levels. |
| | Receptor 1). | Susceptibility marker for pre-eclampsia. |
| | | Frequent and persistent cannabis use, abuse |
| | | and dependence. |
| | | Increased BMI and waist circumference. |
| | | Addiction and other psychiatric disorders. |
| rs806370, and | CNR1 (Cannabinoid | Serum leptin levels. |
| rs12720071. | Receptor 1). | |
| rs140700. | SLC6A4 (Serotonin | Interaction with the cannabinoid receptor 1 |
| | transporter). | gene promoter was significantly associated |
| | | with anxious phenotype. |
| rs1019238, and | ANKFN1 (Ankyrin | Cannabis dependence. |
| rs1431318. | Repeat And | |
| | Fibronectin Type III | |
| | Domain Containing 1) | |
| rs806381. | CNR1 (Cannabinoid | Visceral fat mass. |
| | Receptor 1). | Significant association with triglyceride. |
| rs806378. | CNR1 (Cannabinoid | Weight gain in patients of European ancestry |
| | Receptor 1). | treated with clozapine or olanzapine. |
| | | Fat distribution and abdominal adiposity in |
| | | men. |
| rs1049353, and | CNR1 (Cannabinoid | Susceptibility to mood disorders. |
| rs324420. | Receptor 1) and | |
| | FAAH (Fatty Acid | |
| | Amide Hydrolase). | |
| rs2295632, and | FAAH (Fatty Acid | Early onset obesity. |
| rs324420. | Amide Hydrolase). | |
| rs6454674, | CNR1 (Cannabinoid | Alcohol dependence. |
| rs1049353, and | Receptor 1). | |
| rs806368. | | |
| rs806365. | CNR1 (Cannabinoid | Insulin resistance |
| | Receptor 1). | |
| rs6454674. | CNR1 (Cannabinoid | Cocaine dependence. |
| | Receptor 1). | |
| rs806377, and | CNR1 (Cannabinoid | Association with differential gaze duration |
| rs806380. | Receptor 1). | for happy faces. |
| rs41311993. | CNR2 (Cannabinoid | Bipolar disorder. |
| | Receptor 2). | |
| rs806379. | CNR1 (Cannabinoid | Latency of psychosis after the first |
| | Receptor 1). | consumption of methamphetamine. |
| rs9444584. | CNR1 (Cannabinoid | Strong linkage disequilibrium with |
| | Receptor 1). | rs9450898 and rs2023239. |
| rs9450898. | CNR1 (Cannabinoid | Addiction and obesity as a result of cannabis |
| | Receptor 1). | misuse. |
| rs4253765, | PPARA (peroxisome | Schizophrenia. |
| rs4263776, | proliferator activator | |
| rs6007662, | receptor). | |
| rs1800206, | | |
| rs4253763, | | |
| rs6008197, and | | |
| rs4253655. | | |
| rs1049353, | CNR1 (Cannabinoid | Association with self-reported smoking and |
| rs12720071, and | Receptor 1). | susceptibility markers for specific dietary |
| rs806368. | | composition. |
| rs6454672. | CNR1 (Cannabinoid | Decreased likelihood of meeting physical |
| | Receptor 1). | activity recommendations. |
| rs324419, | FAAH (Fatty Acid | Parkinson disease related pain. |
| rs2295633, and | Amide Hydrolase). | |
| rs6746030. | | |
| rs2180619. | CNR1 (Cannabinoid | G allele has been associated with addiction |
| | Receptor 1). | and high levels of anxiety, as risk for |
| | | ‘psychopathological conditions'. |
| rs35761398. | CNR2 (Cannabinoid | Severe inflammation and hepatocellular |
| | Receptor 2). | necrosis in HCV patients. |
| rs1978340, and | GAD1 (Glutamate | Strong association with heroin dependence. |
| rs3791878. | decarboxylase 1) | |
| rs3123554. | CNR2 (Cannabinoid | Body weight, triglyceride, HOMA-IR, |
| | Receptor 2). | insulin, and leptin levels. |
| rs760288. | NRXN3 (neurexin 3). | Alcohol dependence. |
| rs806365, | CNR1 (Cannabinoid | Poorer anxiety treatment response. |
| rs7769940, | Receptor 1), FAAH | |
| rs2209172, | (Fatty Acid Amide | |
| rs2501431, and | Hydrolase) and CNR2 | |
| rs2070956. | (Cannabinoid | |
| | Receptor 2). | |
| rs806366. | CNR1 (Cannabinoid | Predisposition to headache with nausea in the |
| | Receptor 1). | presence of life stress. |
| rs806368, | CNR1 (Cannabinoid | Decreased volume of the right anterior |
| rs1049353, | Receptor 1). | cingulum with cannabis exposure. |
| rs2023239, and | | |
| rs6454674 | | |
| rs1406977, and | CNR1 (Cannabinoid | Lower cortical response. |
| rs20417. | Receptor 1), and | |
| | PTGS2 | |
| | (prostaglandin- | |
| | endoperoxide | |
| | synthase). | |
| rs806374. | CNR1 (Cannabinoid | Individual differences in level of cannabis |
| | Receptor 1). | use over time. |
| rs806372, | CNR1 (Cannabinoid | Extraversion, and modulation of personality |
| rs2180619, | Receptor 1). | and psychiatric conditions. |
| rs806368, and | | |
| rs806371. | | |
| rs1799971, | OPRMI (Opioid | Hypoalgesic responses. |
| rs4680, | Receptor Mu Subunit), | |
| rs324420. | COMT (catechol-O- | |
| | methyltransferase) and | |
| | FAAH (Fatty Acid | |
| | Amide Hydrolase). | |
| rs2501431, and | CNR2 (Cannabinoid | Increased osteoporosis risks. |
| rs3003336. | Receptor 2). | |
| rs1049353, | CNR1 (Cannabinoid | Cannabis use disorder. |
| rs806378, and | Receptor 1), and | |
| rs324420. | FAAH (Fatty Acid | |
| | Amide Hydrolase). | |
| rs2501432. | CNR1 (Cannabinoid | Remarkable mark for depression. |
| | Receptor 1). | |
| rs806368, and | CNR1 (Cannabinoid | No significant association with |
| rs17228602. | Receptor 1), and | acetylcholinesterase activity in cannabis |
| | ACHE | addicted and non-addicted healthy controls. |
| | (Acetylcholinesterase). | |
| rs806368, | CNR1 (Cannabinoid | Arsenic concentration in urine, lead |
| rs806381, | Receptor 1). | exposure. |
| rs1049353, and | | |
| rs12720071. | | |
| rs806374, | CNR1 (Cannabinoid | Psychiatric comorbidities in Anorexia |
| rs3003335, and | Receptor 1), and | Nervosa patients. |
| rs6658703. | CNR2 (Cannabinoid | |
| | Receptor 2). | |

Accordingly the present technology can be used to determine a patient's risk for administration of a cannabinoid containing medicine. For example by testing for any one or more of the above SNPs, either alone or in combination with other SNPs, and providing a medicine recommendation accordingly.

In addition to using genetic information to determine user risk for administration of cannabinoid containing medicines, the analysis of a user's genetic information can be used to determine or rule out potential sources of risk, or side effects. For example the presence of one or more of the SNPs identified herein can be used to indicate a low risk of the related risk factor.

| Single Nucleotide | | |
|---|---|---|
| Polymorphisms (SNPs) | Gene | Risk Factor |
| rs9353527, | CNR1 | No evidence for association with obesity in |
| rs754387, | (Cannabinoid | German children. |
| rs6454676, | Receptor 1). | |
| rs806379, | | |
| rs1535255, | | |
| rs2023239, | | |
| rs806370, and | | |
| rs1049353 | | |
| rs1049353, | CNR1 | Inconclusive evidence of association with the |
| and | (Cannabinoid | development of cannabis dependence. |
| rs806380. | Receptor 1). | |
| rs2023239. | CNR1 | No association with increased overweight and |
| | (Cannabinoid | obesity risk. |
| | Receptor 1). | |
| rs806381. | CNR1 | No association with increased overweight and |
| | (Cannabinoid | obesity risk. |
| | Receptor 1). | |
| rs324420. | FAAH (Fatty Acid | Protective against anorexia nervosa. |
| | Amide Hydrolase). | No association with IBS pathogenesis. |
| rs6928813. | CNR1 | Favourable anxiety treatment response. |
| | (Cannabinoid | |
| | Receptor 1). | |
| rs1049353. | CNR1 | Lower body mass index, fat mass, and insulin |
| | (Cannabinoid | levels. |
| | Receptor 1). | No association with susceptibility to depression. |
| | | No contribution to susceptibility to cannabinoid |
| | | addiction in a Turkish population. |
| | | No association with weight gain after |
| | | antipsychotic exposure. |
| | | No association with anorexia nervosa. |
| | | No association with cognitive impairment in |
| | | multiple sclerosis patients. |

It should be appreciated that the foregoing single nucleotide polymorphisms are provided by way of example only, and the systems and methods described herein are designed to incorporate further genotype information as their effects on pharmacokinetics and medicine use become known.

### 6.6.2. Dosing Factor Information

The inventors are developing representative base-line dosing factor information 506 for a range of clinical information in accordance with the present technology. It should be appreciated that these values may be updated over time in accordance with the present technology. Where specific values are not provided in the tables, the values may be inferred by interpolating between the values provided.

It should be understood that the dosing levels described herein are based on the inventors' best knowledge and understanding. It should also be appreciated that a recommended dosage may be varied for some reason to account for patient specific clinical information. It should be understood that the dosage factors described herein may not be appropriate for all patients. It is recommended that patients consult a clinician before relying on the information described in this specification.

Based on current clinical information, the inventors recommend that cannabinoid containing medicines are administered to a patient, and therefore recommended by the present technology and included in a prescription, as:
1 mg to 2.5 mg of THC per day, and to be titrated to a maximum daily dose of 30 mg per day; and
5 mg to 10 mg of CBD per day, and to be titrated to between 200 mg to 300 mg per day.

Table 1 provides an example of potential total daily limits for CBD and THC dosages to treat a range of medical conditions for a nominal patient. Also included are recommended starting dosages for both CBD and THC. These values are based on the inventors' current understanding.

TABLE 1
Total Daily Limits and Starting Dosages for CBD and THC

| | | Total daily limits (mg) | | | | |
|---|---|---|---|---|---|---|
| | Patient age | CBD | | THC | | Doses/ |
| Condition | category | Start | Max | Start | Max | day |
| Epilepsy | Adult | 100 | 5000 | 2.7 | 27 | 2 |
| | Pediatric | 5 | 200 | 0.5 | 5 | 2 |
| Spasticity | Adult | 2.5 | 25 | 2.7 | 27 | 2 |
| | Pediatric | 1.25 | 12.5 | 1.35 | 13.5 | 2 |
| Pain | Adult | 50 | 300 | 2.5 | 30 | 2 |
| | Pediatric | 5 | 200 | 0 | 0 | 2 |
| Acute pain | Adult | 50 | 300 | 2.5 | 30 | 2 |
| | Pediatric | 5 | 200 | 0 | 0 | 2 |
| Insomnia | Adult | 50 | 300 | 2.5 | 30 | 2 |
| | Pediatric | 5 | 200 | 0 | 0 | 2 |
| Anxiety | Adult | 50 | 300 | 2.5 | 30 | 2 |
| | Pediatric | 5 | 200 | 0 | 0 | 2 |
| Autism | Adult | 50 | 300 | 2.7 | 30 | 2 |
| | Pediatric | 5 | 200 | 1.35 | 13.5 | 2 |
| PTSD | Adult | 30 | 300 | 2.7 | 27 | 2 |
| | Pediatric | 1.25 | 30 | 1.35 | 13.5 | 2 |
| Tourette | Adult | 30 | 300 | 2.7 | 27 | 2 |
| | Pediatric | 1.25 | 12.5 | 1.35 | 13.5 | 2 |
| Cachia | Adult | 30 | 300 | 2.7 | 27 | 2 |
| | Pediatric | 1.25 | 12.5 | 1.35 | 13.5 | 2 |
| Nausea | Adult | 30 | 300 | 2.7 | 27 | 2 |
| | Pediatric | 1.25 | 12.5 | 1.35 | 13.5 | 2 |
| Parkinson's | Adult | 30 | 300 | 2.7 | 27 | 2 |
| | Pediatric | na | na | na | na | na |
| Fibromyalgia | Adult | 50 | 300 | 5 | 30 | 2 |
| | Pediatric | na | na | na | na | na |

In examples of the technology, a patient diagnosed with a medical condition, such as those listed in Table 1, may be prescribed a medication comprising CBD and/or THC. This medication is optionally administered in small doses, and gradually increased until the desired therapeutic effect is achieved. For example, an adult patient suffering from anxiety, may be prescribed a medication comprising a 10:1 ratio of CBD to THC. In order to reduce the likelihood of negative side-effects occurring, the patient may be administered a low-dosage of the medication, which is gradually increased, for example over a four week period until the desired therapeutic effect is achieved.

In examples of the technology, one or more of the total daily limits, starting doses and ratio of CBD to THC may be modified in accordance with clinical and or genetic information about the patient. For example, where a patient is found to have a low THC metabolism rate, the corresponding THC dosage should be reduced proportionally e.g. 40% thereof. For patients where multiple factors apply, each factor should be taken into account to calculate a recommended daily dosage e.g. each factor may be multiplied to the base-line dosage level to calculate the recommended dosage.

### 6.6.2.1. Cannabinoid Specific Dosage Factors

Table 2 provides example dosage factors for a nominal patient having a Single Nucleotide Polymorphism (SNP) in the listed genes. It should be understood that the nature of the SNP may provide an indication as to the patient's likely risk factor/metabolism rate. In other words, Adenine (A), Thymine (T), Cytosine (C), and Guanine (G) nucleotide substitutions in a given gene may provide different information as to a patient's likely risk or metabolism rate for a given medication.

TABLE 2
SNP Dosage Factors

| | | Dosage |
|---|---|---|
| Factor | Indication | Factor |
| THC Metabolism Rate | High Metabolism | 1.0 |
| (CYP2C9 SNP) | Medium | 0.48 |
| | Metabolism | |
| | Low Metabolism | 0.4 |
| Psychosis Risk (AKT1 SNP) | High Risk | 0.1 |
| | Medium Risk | 0.4 |
| | Low Risk | 1 |
| Neurocognitive Impairment | High Risk | 0.2 |
| Risk (COMT SNP) | | |
| | Medium Risk | 0.6 |
| | Low Risk | 1 |
| CBD Metabolism Rate | Very High | 1.5 |
| (CYP2C19 SNP) | High | 1.25 |
| | Medium | 1 |
| | Low | 0.75 |
| | Very Low | 0.5 |

It should also be understood that if a patient has multiple risk factors for an adverse side affect from administration of a cannabinoid compound, that the dosage factor for that compound (or a varin analogue) may be zero. For instance, if the patient has both a high risk of neurocognitive impairment from THC (COMT SNP) and high risk of physchosis (AKITT SNP) that the dosage factor is substantially zero. Alternatively or in addition, the dosage factor for a compound may be substantially zero if assessment of an SNP indicates the patient has an addiction risk form administration of that compound.

Table 3 provides example dosage factors for nominal patients that have previously been prescribed cannabinoid containing medications or who have consumed cannabinoid containing substances recreationally.

TABLE 3
Cannabis Use Dosage Factors

| Factor | Indication | Dosage Factor |
|---|---|---|
| Past Cannabis User | Yes | 1.5 |
| | No | 1.0 |
| Plasma THC Concentration | 0 | 1.0 |
| (ng/ml) | 5 | 0.75 |
| | 10 | 0.5 |
| | 15 | 0.25 |
| | 20 | 0 |
| Plasma CBD Concentration | 0 | 1.0 |
| (ng/ml) | 5 | 0.75 |
| | 10 | 0.5 |
| | 15 | 0.25 |
| | 20 | 0 |

The THC and CBD concentrations in a patient's plasma can also be used to determine if the patient is metabolising a component of the cannabinoid containing medicine at a target rate during a course of medicine. For instance, that can be used to double check that metabolism is occurring as predicted according to the SNPs identified during a genetic test and which form part of the patient information. It can therefore be advantageous to feed these measurements to the feedback system 108 to optimise the recommendations provided by the processing system 104.

### 6.6.2.2. Base Biological Dosage Factors

Table 4 provides example dosage factors based on representative biological factors for nominal patients.

TABLE 4
Biological Factors

| Factor | Indication | Dosage Factor |
|---|---|---|
| Sex | Male | 1.0 |
| | Female | 0.8 |
| Age (Years) | 1 | 0.2 |
| | 5 | 0.4 |
| | 10 | 0.56 |
| | 15 | 0.73 |
| | 20 | 0.85 |
| | 25 | 0.9 |
| | 30 | 1.0 |
| | 35 | 0.95 |
| | 40 | 0.9 |
| | 45 | 0.85 |
| | 50 | 0.8 |
| | 55 | 0.75 |
| | 60 | 0.7 |
| | 65 | 0.65 |
| | 70 | 0.63 |
| | 75 | 0.6 |
| | 80 | 0.58 |
| | 85 | 0.6 |
| | 90 | 0.62 |
| Weight (Kg) | 10 | 0.4 |
| | 15 | 0.45 |
| | 20 | 0.5 |
| | 25 | 0.55 |
| | 30 | 0.6 |
| | 35 | 0.65 |
| | 40 | 0.7 |
| | 45 | 0.75 |
| | 50 | 0.8 |
| | 55 | 0.85 |
| | 60 | 0.9 |
| | 65 | 0.95 |
| | 70 | 1.0 |
| | 75 | 1.05 |
| | 80 | 1.1 |
| | 85 | 1.15 |
| | 90 | 1.2 |
| | 95 | 1.25 |
| | 100 | 1.3 |
| | 105 | 1.35 |
| | 110 | 1.4 |
| | 115 | 1.45 |
| | 120 | 1.5 |
| Fat Content (%) | 6 | 1.25 |
| | 10 | 1.2 |
| | 14 | 1.15 |
| | 18 | 1.1 |
| | 22 | 1.05 |
| | 26 | 1.0 |
| | 30 | 0.95 |
| | 34 | 0.9 |
| | 38 | 0.85 |
| | 42 | 0.8 |

### 6.6.2.3. Organ Function Based Dosing Factors

Table 5 provides representative dosing factors for a patient who has reduced kidney or liver function.

TABLE 5
Organ Function Dosage Factors

| Factor | Indication | Dosage Factor |
|---|---|---|
| Kidney Function | Normal | 1.0 |
| | Low | 0.5 |
| | Very Low | 0 |
| | Critical | 0 |
| Liver Function (%) | Normal | 1.0 |
| | Low | 0.5 |
| | Very Low | 0 |
| | Critical | 0 |

### 6.6.2.4. Composition Examples

In many situations it may be necessary to optimise the relative amount of two or more active compounds which are administered to a patient. For instance, the present technology may recommend administration to a patient of a specific ratio of two compounds due to the absence or presence of single nucleotide polymorphisms. This could be useful to optimise therapeutic efficacy and reduce or minimise adverse affects e.g. a patient having a lower rate of THC metabolism and a standard CBD metabolism rate would ideally be administered a medicine having less (or potentially zero) THC.

In one example of the technology, the present technology may recommend a medicine from a list of options. Exemplary formulations are provided in Table 6 below, in which the ratio of the actives varies between the formulations:

TABLE 6
Exemplary Ratio of CBD:THC

| Formulation | Ratio of CBD:THC |
|---|---|
| A | 10:0 |
| B | 10:1 |
| C | 10:2 |
| D | 10:3 |

In these embodiments, the present technology may provide a dosage recommendation for one of the formulations using the methods and systems described herein. It should be understood, that the exemplary formulations according to Table 6 may have a fixed concentration of one of the actives, and the concentration of the other may vary to provide the stated exemplary ratios. Alternatively, the concentration of each active may vary between the exemplary formulations of Table 6 in addition to the ratio of actives.

In yet a further example, the present technology may provide a medicine recommendation from a list of options. Exemplary formulations are provided in table 7 below, in which the ratio of actives is the same in each formulation but the concentration differs:

TABLE 7
Exemplary Formulation Strengths

| Formulation | CBD Concentration | THC Concentration |
|---|---|---|
| Example | (mg/ml) | (mg/ml) |
| W | 10 | 1 |
| X | 50 | 5 |
| Y | 100 | 10 |
| Z | 300 | 30 |

This approach may be beneficial to assist in reducing the number of dosages that need to be administered to a patient to achieve a given therapeutic effect. Alternatively, or in addition, it may provide additional flexibility in prescribing medicines to patients. In yet a further example, the present technology may provide recommendations on two or more medications and dosages of each. For instance, the list of medicines may comprise one or more formulations having a first active compound, and one or more formulations containing a second active. Exemplary formulations for use in this embodiment are outlined in Table 8 below:

TABLE 8
Exemplary Formulations

| First | Concentration CBD | Second | Concentration THC |
|---|---|---|---|
| medication | (mg/ml) | medication | (mg/ml) |
| A1 | 10 | A2 | 1 |
| B1 | 50 | B2 | 5 |
| C1 | 100 | C2 | 10 |
| D1 | 300 | D2 | 30 |

In these embodiments, the technology is configured to provide a recommendation for a first medicine containing the first active and a second medicine containing the second active. The recommendation may provide a specific dose of each of the first medicine and the second medicine. For instance, the technology may recommend one or more of the following:
one of formulations A1, B1, C1 and D1;
one of formulations A2, B2, C2 and D2;
the amount of the first medication to be administered; and
the amount of the second medication to be administered.

This approach to providing recommendations may provide greater flexibility in prescribing medications to patients. In addition, it may better assist in prescribing medications to meet an individual patient's medical needed e.g. according to genetic criteria, medical requirements, and other factors as described herein.

It should also be understood, that in embodiments the present technology may be configured to update the recommendation for one of more of the first medication and the second medication e.g. based on patient feedback provided using the systems and method described herein, or an improved population model indicting that a recommendation or dosage could be improved.

### 6.6.2.5. Dosage Regimes

One method of administering a medication to minimise the likelihood of negative side effects is to start with a low initial dose and gradually increase the dosage over time until the desired therapeutic effect is achieved. This may be particularly beneficial for prescribing and administering medications with a narrow therapeutic index i.e. a medication which has a narrow separation between the dosage which provides the desired therapeutic effect and the dosage which provides negative side effects. Examples of medications with a narrow therapeutic index include (but are not limited to) Warfarin, Levothyroxine and Digoxin.

One downside to the prior art methods is that it can take a long time for a therapeutic effect to be achieved. This is particularly detrimental for high cost medications, or the patient is experiencing severe symptoms which require immediate relief.

In examples, the present technology can partially or completely address the downsides of the prior art by applying the dosing factors described herein to provide improved medicine recommendations e.g. to optimise one or more of the initial dose recommendation, rate of dosage increase and maximum dosage in order to arrive at the therapeutic level faster.

In yet further examples of the present technology, population data or a population model as described herein may be used to predict an initial dose which is likely to provide the desired therapeutic effect. In this way the delay before receiving a therapeutic effect can be minimised, while maintaining a low chance of experiencing negative side effects. For example, the population data or model may suggest based on genetic and/or clinical information of similar patients that a given dosage would provide the desired therapeutic effect. The initial dose can then be set at the recommended dosage or alternatively slightly below the recommended dosage to further reduce the likelihood of negative side effects developing.

### 6.6.2.6. Medication Selection

In one example of the present technology, the dosing factors and/or population data/models described herein may be used to optimise the medication recommended to a patient.

For example, where a patient suffers pain, such as neuropathic pain, it can be common to start the patient on a first medication, and if the medication is found to be ineffective in treating the symptoms, the patient can be moved onto a second, third or fourth medication. For example, a patient may first be prescribed a treatment comprising paracetamol, and if the treatment is found to be ineffective, the prescription may be changed to a stronger medication such as codeine. This process can require trialing multiple medications until an appropriate therapeutic effect is achieved.

In contrast, the present invention can reduce the trial and error approach to prescribing medications by using clinical and/or genetic information to recommend a medication which is mostly likely to provide the desired therapeutic effect in the first instance while minimising the likelihood of negative side effects occurring.

### 6.6.3. User Interface

An example of a user interface and method of interacting with a device 700 according to the present technology are illustrated in FIGS. 7a to 7e.

Referring firstly to FIG. 7a which shows an example credential validation interface for the present technology. As shown the interface prompts the patient to provide identifying credentials such as their name, date of birth, sex and a unique identifier (in this case a national health index number). These details are used to confirm the identity of the patient, and in some cases extract information about the patient from one or more databases.

For example, the device 700 may be configured to communicate with a practise management system in order to extract details about the patient, such as their medical history. In other examples, the credentials may be used to access details on the device itself, or a server configured to track the patient's use of the recommendation system. In other examples, the patient may be prompted to provide log-in credentials, as should be familiar to those skilled in the art.

Once the details have been entered, the patient is prompted to confirm that the details are correct before proceeding.

FIG. 7b shows a confirmation screen where the patient's medical history has been downloaded from a database. These details may be confirmed if correct, or modified if incorrect or incomplete.

If the device 700 fails to download the patient's medical history, the patient may instead be prompted to enter a list of existing medical conditions as shown.

FIG. 7c shows an interface which prompts the patient to input details about the condition requiring treatment. This may include both the cause of the injury if applicable, the main symptom to be treated, as well as any additional symptoms requiring treatment.

In other examples of the technology, the patient may select the symptoms to be treated only, particularly in situations where the underlying cause is unknown.

FIG. 7d. shows a further interface prompting the patient to input additional information such as their height, weight, current medications, past Cannabis usage history, genotype information (if known) and current CBD and THC blood plasma concentrations.

One or more of the above fields may be automatically populated from the database the device connects to in FIG. 7b. It should be appreciated that some of the information shown may not be known. In these cases, the fields may be left blank.

Where genotype information is provided as a standalone file, it may be possible to import these files to extract the required information to populate the form.

FIG. 7e shows an interface which provides a recommendation to the patient as to which medication should be taken to treat the specific medical condition disclosed. This recommendation takes into consideration the clinical information provided to recommend a product or strain (when the recommendation relates to cannabinoid containing medicines), as well as a specific dose of active ingredient.

Note that in the example provided, the administration route information is provided as the specific route affects pharmacokinetics of the drugs involved. Accordingly, administration route recommendations may be used to control blood plasma levels of CBD/THC in addition to simply varying the dosage strengths and relative concentrations.

In some examples of the technology, the device 700 may be configured to automatically generate a prescription, or alternatively send a request for a prescription based on the recommendations provided.

In some examples of the technology, the device 700 may also be configured to provide information to the prescriber, patient/user such as regulatory information, warnings, and/or prompt for informed consent. For example, the device 700 may be configured to provide information relating to medications which fall within section 29 of the New Zealand Misuse of Drugs Act 1975.

### 6.6.4. Updating the Processing Systems

One example of how a processing system 104 can be updated to provide improved recommendations is by updating the dosage factor information 506 based on the feedback provided. For example, if two patients, patient A and patient B are looking to treat the same symptoms, and have similar clinical information. Feedback from patient A on the efficacy and side-effects of a particular drug can be used to influence the recommendations for patient B (for example drug X was ineffective, but drug Y was effective).

Where there are differences between patient A and patient B's clinical information (i.e. biological, pharmacogenetic and/or pharmacokinetic information), these differences can be used to fine-tune the dosage factors. For example, if patient A shares the same clinical information as patient B but is 20 kg lighter differences in therapeutic efficacy or side-effects experienced may be attributable to the weight difference. Accordingly, the dosage scaling based on weight can be increased or decreased as necessary to result in a similar treatment outcome.

In some examples, the feedback system 108 may also incorporate test results. For example, after a period of treatment (such as one month), the patient may be tested to determine the drug concentrations present in the patient's system. For example, the patient's blood plasma may be tested to determine drug concentrations. Where drug concentrations are higher than expected, this may indicate the processing system 104 is overestimating the patient's ability to metabolise the prescribed drug. Accordingly, the processing system 104 may recommend a lower dose of active ingredient to reduce build-up in the patient's system.

The rate at which the processing system 104 update's its dosage factor information 506 can depend on a number of factors. For example, where large clinical information differences exist between patients, it may be difficult to attribute any inequality of outcome with any specific factor, accordingly adjustments should be minor (or in some cases no adjustments should be made).

In examples where only a few differences exist between patients, and the differences can be attributed to one or more parameters, larger adjustments may be made.

Consideration should also be given to the amount of information present in the processing system, for example the number of patient records, or the amount of population data. For example, if 100 people have similar clinical information and similar experiences on a given medication, then having a single outlier who has a different experience should not necessarily materially influence the dosage factor information 506. Minimising the effects of outliers can be done in a number of ways, as those familiar with statistics can appreciate. For example, results which are significant outliers can be ignored, or the variation in efficacy/side-effects may be averaged across the number of respondents.

### 6.6.4.1. Artificial Intelligence

Given the amount of clinical information, medicine information 502, dosage factors 506, and optionally price and availability information 504 being processed by the present technology, it may be advantageous for one or more systems described herein to be optimised using an artificial intelligence, such as machine learning.

### 6.6.4.2. Dosage Factors

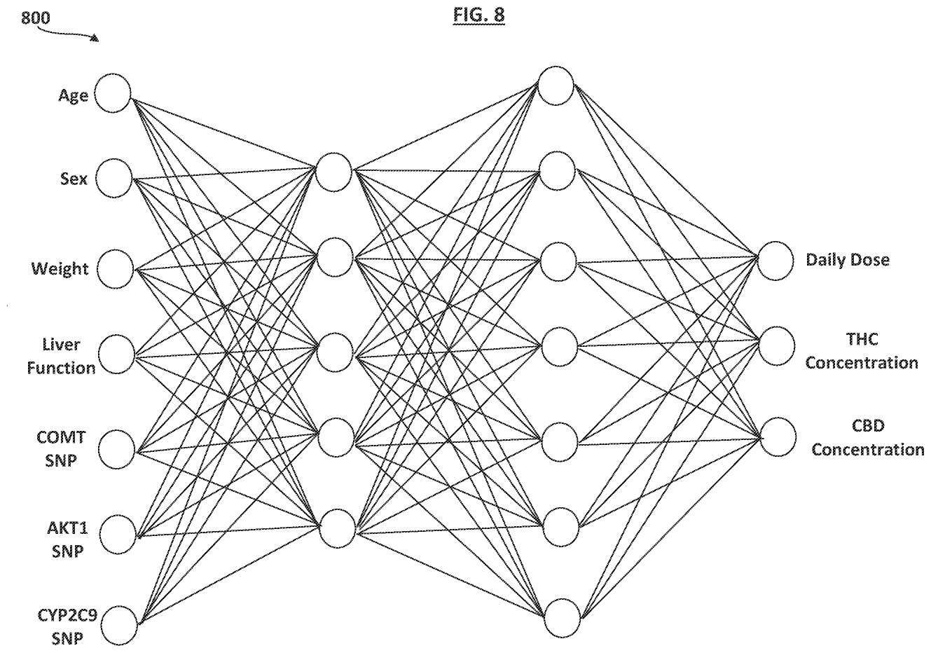

One method of updating the dosage factors for the present technology is to utilise machine learning to process large sets of data such as patient records or population data as described herein. For example, as shown in FIG. 8 a neural network 800 may comprise a series of neurons or nodes which represent the clinical information or patient records. For example, age, weight, height, genomic SNP data, and organ function information. The neural network 800 can then be configured to output dosage recommendations and concentration information for the active ingredient (such as CBD and THC) concentrations. These recommendations can then be compared against a database of known products to find the closest match to the composition recommended by the neural network 800.

For sake of simplicity the neural network pictured has been simplified by only showing a selection of the clinical information which impacts the recommendations provided.

While the neural network 800 may be initialised with random weighting or bias values. It can be advantageous for the neural network to be initialised with the specific dosing factors disclosed herein. In this way the neural network 800 may optimise the recommendations (by tweaking the weightings and biases to minimise the cost function) in fewer iterations than may otherwise be required.

The neural network 800 may undergo a learning routine based on a history of past medical recommendations. This can either be done on as supervised learning or reinforcement learning.

For example, training data may comprise clinical information which is coded to also indicate what the prescribing physician recommended for treatment. In other examples, a physician may review the recommendations made by the neural network either during or after the learning phase in order to ensure that the recommendations made are correct.

It should be appreciated that some parameters may be excluded from the neural network 800 and for instance have set factors programmed. For example, the influence of cost and availability on the recommendation provided may have a low fixed weighting in order to ensure that the recommendations place more weight on prescribing the correct medication to treat the symptoms, rather than the cheapest.

Once the learning phase has been completed the population model generated by the neural network 800 should be validated against a different dataset than the trained data. This can help to ensure that the model is behaving as expected.

In use the model can either be kept static, or updated during use. In one example the recommendation system may comprise a static model which has been optimised by machine learning. In another example, the recommendation system may be periodically updated as additional patient records or population data is obtained. In a yet further example, the model may be updated as each recommendation is made.

In a yet further example of the technology the model generated by the neural network 800, outputs a lookup table of dosage factors which can be used by the systems described herein to provide medicine recommendations. This dosage factor table can be updated periodically, as new data is added, as recommendations are made, manually, or using any other technique known to those skilled in the art.

### 6.6.4.3. Medicine Information

Given the large amount of medical literature in circulation and the rate at which new findings are being published, it may be advantageous for an AI system to be employed to identify relevant medical publications which relate to the specific medicines being recommended by the present technologies. For example, an AI system may be trained to look for occurrences of specific phrases within medical journals or words (such as the active ingredients of the medicines being recommended) which are used in conjunction with other relevant terminology such as efficacy or side-effects.

For example, the AI system may perform natural language processing on the medical journals to determine relevance to the medicines being recommended herein.

Once identified, a person may manually review each article for relevance, and extract any pertinent information to the recommendation systems described herein. This may result in an update to the medicine information used to provide recommendations. The reviewer's findings may also be fed back to the AI system in order to provide reinforcement learning which may improve future identification of relevant information. For example, where a neural network is used (such as the one shown in FIG. 8) the reviewer's findings may be used to back-propagate the results through the neural network.

Accordingly, when significant changes are made to the medicine information it may be appropriate for the neural network such to be retrained to account for the new information.

### 6.6.4.4. AI Models

The present technology may be implemented using any appropriate AI or neural network model known to those skilled in the art. For example, the open-source machine learning platform Tensorflow™ may be used.

### 6.6.5. Systems, Methods and Devices

In the examples the technology described herein relates to devices, systems and methods of providing medicine recommendations.

### 6.6.5.1. Recommendations and Prescriptions Based on Genetic Testing

One method of recommending a cannabinoid containing medicine to administer to a patient comprising:
A) testing at least one genetic sample from the patient to determine the presence or absence of at least one single nucleotide polymorphism (SNP) associated with one or more of the patient's possible psychosis risk caused by administration of THC and the patient's risk of neurocognitive impairment caused by administration of THC;
B) testing at least one genetic sample to assess at least one SNP associated with metabolism of one or more of cannabidiol (CBD) and tetrahydrocannabinol (THC); and
C) providing an indication of the patient's suitability to be administered a cannabinoid containing medication based on the assessed SNPs.

It should be appreciated, that where the genetic information is already available, tasks A and B may be excluded, and a recommendation may be provided using the systems and methods described herein.

In yet further examples of the technology, where no genetic information is available, the patient may be asked to have genetic testing done in order to determine their suitability to receive medications containing cannabinoids.

In other examples, such as where genetic testing is not available or otherwise prohibitive for the patient (i.e. requires travel or is prohibitively expensive), the systems and methods described herein may be configured to provide medicine recommendations without any genetic information.

For example, the processing system 104 may provide recommendations without any genetic information by comparing the outcomes of similar recommendations made in the past. In other examples, the processing system 104 may be configured to assume that where no genetic information is provided, the patient is likely to have the genetic composition which is most common among previous patients. In a yet further example, the processing system may be configured to assume worst-case SNP information (i.e. slow metabolism of CBD and/or THC, and high-risk factors for neurocognitive impairment or psychosis) to thereby provide conservative recommendations.

### 6.6.5.2. Recommendations and Prescriptions Based on Genetic Information

In another example the technology provides recommendations based on genetic information provided by the patient or the patient's prescribing physician. For example, the present technology provides for systems and devices configured to provide a recommendation for a medicine for administration to a patient, comprising:
a graphical user interface (GUI);
an input system for receiving at least one piece of genetic information;
wherein the at least one piece of genetic information includes one or more of the patient's possible psychosis risk due to at least one of administration of the medicine and the patient's neurocognitive impairment risk due to administration of the medicine;
the patient's likely metabolism rate of at least one active ingredient of the medicine; and
a processing system that is configured to provide an output indicative of a medicine recommendation for the patient based on the patient's likely metabolism rate of the at least one active ingredient in the medicine in combination with one or more of the patient's possible psychosis risk and neurocognitive impairment risk.

In other examples, there are provided systems configured to generate medicine recommendations for administration to a patient, the systems comprising:
an input system for receiving at least one piece of genetic information;
wherein the at least one piece of genetic information includes one or more of the patient's possible psychosis risk and the patient's neurocognitive impairment risk; and
the patient's likely metabolism rate of at least active ingredient of a medicine; and
a processing system that is configured to provide an output indicative of a medicine recommendation for the patient based on the patients likely metabolism rate with one or more of the patient's possible psychosis risk and neurocognitive impairment risk.

In other examples, the present technology includes methods for providing a recommendation for a medicine to administer to a patient, the methods comprising:
A) receiving information regarding a patient's medical condition or at least one symptom experienced by the patient;
B) receiving patient information comprising physiological and genetic information;
C) providing dosage recommendations based on the patient information.

In the foregoing examples it should be appreciated that the metabolism rates, neurocognitive impairment risk and psychosis risk may be associated with cannabinoid containing medicines as described herein.

### 6.6.5.3. Automated Generation of Prescriptions

One benefit of the present technology is the ability to provide automated prescriptions for the medication(s) recommended to the patient. For example, there is provided a system to generate prescriptions for administration of medicines to a patient, the system comprising:
an input system configured to receive patient information,
a processing system configured to generate medicine recommendations using the patient information; and
an output system configured to generate a prescription containing the medicine recommendation based on the patient information; and
wherein the patient information comprises at least one single nucleotide polymorphism (SNP) associated with the patient's metabolism rate of at least one active ingredient in a medicine in combination with at least one SNP associated with the patient's possible psychosis risk and the patient's neurocognitive impairment risk due to taking the medicine.

In other examples, there is provided a device configured to generate prescriptions for administration of medications to a patient, the device comprising:
an input system configured to receive patient information;
a processing system configured to generate a medicine recommendation using the patient information;
an output system configured to generate a prescription containing the medicine recommendation based on the patient information; and
wherein the patient information comprises at least one single nucleotide polymorphism (SNP) associated with associated the patient's metabolism rate of at least one active ingredient of a medicine in combination with at least one SNP associated with the patient's possible psychosis risk and the patient's neurocognitive impairment risk due to administration of the medicine.

It should be appreciated that prior to generating the prescription, the output system may require input or approval from a prescribing physician for example if the prescription must be provided by a doctor or prescribing physician.

### 6.6.5.4. Automated Provision of Dosage Recommendations

Another advantage of the present technology is the ability to provide a patient with dosage recommendations. These may be provided with composition recommendations (i.e. CBD/THC compositions), but this should not be seen as limiting on the technology. For example, the present technology provides a system for providing medication dosage recommendations for administration to a patient, the system comprising:
an input system configured to receive patient information comprising physiological and genetic information;
a processing system configured to generate a dosage recommendation for a using at least one dosage factor and the patient information; and
an output system configured to provide an output representing the dosage recommendation.

In other examples, the technology provides a device for providing medication dosage recommendations for administration to a patient, the device comprising:
an input system configured to receive patient information comprising physiological and genetic information;
a processing system configured to generate a dosage recommendation for a medicine using at least one dosage factor and the patient information;
an output system configured to provide an output representing the dosage recommendation.

### 6.6.5.5. AI Systems and Methods

Described herein are various systems and methods of providing medicine recommendations using patient records or population data. For example, there is provided a system for providing medicine recommendations for a patient, the system comprising:
an input system configured to receive patient information from a plurality of patients,
a processing system comprising at least one remote application, and
an output system configured to provide the medicine recommendations for a patient based on the patient information,
a feedback system configured to receive clinical feedback of therapeutic effect and symptom control;
wherein the processing system is configured to aggregate patient information from the plurality of patients to create a model of a population, and
further wherein the remote application is configured to modify the model of the population based on the feedback received by the feedback system to create an improved model, and
further wherein the system is configured to provide medicine recommendations to patients based on the improved model.

For example wherein the patient information comprises genetic information including at least one single nucleotide polymorphism as described herein.

While the foregoing example refers to remote applications, this should not be seen as limiting on the technology, and in other examples the processing system may include a local application as described herein. For example, the local application may be configured to provide recommendations based on the model. The model may be updated as local application versions are released, or during operation of the local application, for example by downloading population data or patient records from a remote server.

### 6.6.5.6. Electronic Devices

The methods and systems herein may be performed using devices such as electronic devices. For example, there is provided a system for providing medicine recommendations, comprising:
an input system consisting of at least one electronic device, configured to receive patient information;
a processing system comprising at least one processor;
a computer readable storage medium having computer readable program code embodied herewith and executable by the at least one processor; and
the computer readable program code being configured to provide medicine recommendations based on the patient information and at least one dosage factor.

### 6.6.5.7. Dosage Example

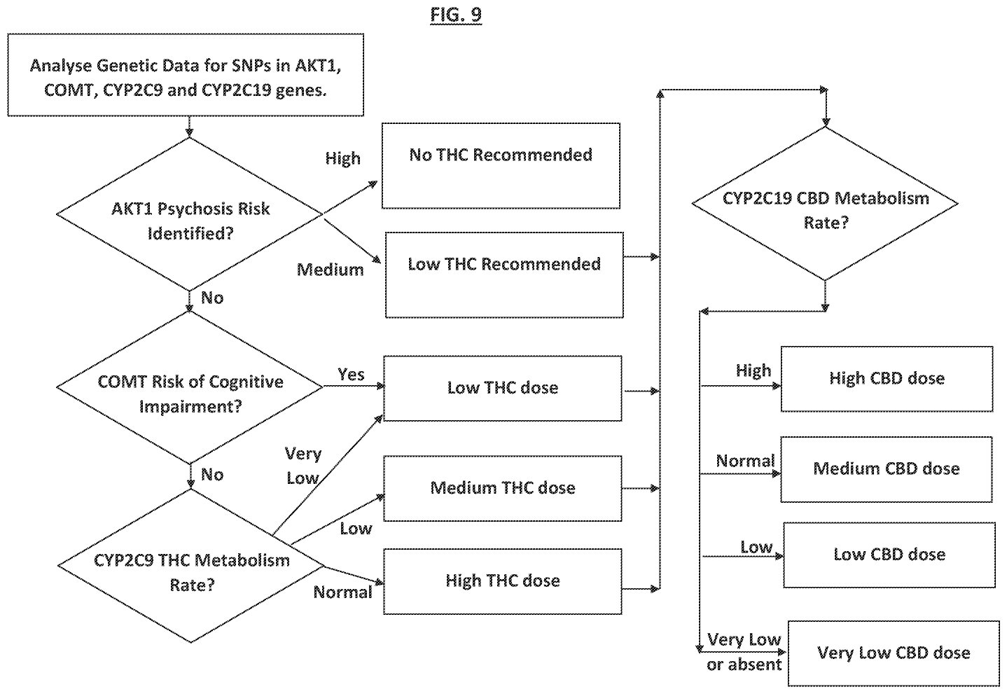

Referring now to FIG. 9 which shows a flow diagram for a further example of the technology, wherein THC and CBD dosage rates may be determined by analysing the COMT, AKT1, CYP2C9, CYP2D6, CYP3A4, CYP1A2 and CYP2C19 genes for single nucleotide polymorphisms (SNPs). It should be appreciated that the foregoing analysis is by way of example only, and in other applications of the technology other SNPs and associated dosage recommendations may be used.

The method involves determining whether the patient has a psychosis risk, optionally by assessing at least the AKT1 and COMT SNPs. If a high psychosis risk is identified, the processing system 104 is configured to recommend a Cannabis dosage having no THC content. If a medium psychosis risk is identified, the processing system 104 is configured to recommend a Cannabis dosage having a medium to low THC content.

If no psychosis risk is found, SNPs are analysed to determine cognitive impairment risk, such as the COMT SNPs described herein. If a risk is identified, the processing system 104 is configured to recommend a low THC dose. In some examples, a cognitive impairment risk may be identified even where no corresponding SNP is identified, for example an elderly person may be considered to have a driving impairment risk from administration of THC, in which case a low to zero THC dose is recommended.

If no cognitive impairment risk is identified, SNPs are analysed to determine a likely rate of THC metabolism. For example, the CYP2C9 SNP may be analysed as described herein. If a very low metabolism rate is detected, a low THC dose is recommended. If a low metabolism is detected, a medium THC dose is recommended. If a normal metabolism is detected, a normal THC dose is recommended.

Once the THC dose has been determined, the system looks at SNPs indicative of the patient's likely CBD metabolism rate, such as CYP2C19. A high rate results in a high CBD dose, a normal rate results in a normal CBD dose, a low rate results in a low CBD dose, and a very low or absent CBD metabolism results in a very low CBD dose. Where the CYP2C19 SNP indicates that the patient is likely to be a superfast metaboliser of CBD an even higher dose may be administered.

In an alternative example, where analysing the AKT1 SNP identifies a possible risk of psychosis, one option is to recommend a low dose of THC comprising less than 1 mg of tetrahydrocannabinol per day. In another example, the system may recommend up to three doses a day of a cannabinoid containing medicine comprising 1 mg of THC or less.

In yet a further example, one or more single nucleotide polymorphisms (SNPs) can be assessed to determine whether a patent has a possible addition risk. For example, the gene encoding for fatty acid amide hydrolade (FAAH), or other SNP, may be assessed to determine a patient's potential addiction risk due to administration of a compound(s). Where an addiction risk is identified, the system can recommend a zero dose of the compound(s) which may cause addiction.

### 6.7. Disclaimer

The foregoing technology may be said broadly to consist in the parts, elements and features referred to or indicated in the specification of the application, individually or collectively, in any or all combinations of two or more of said parts, elements or features.

Aspects of the present technology have been described by way of example only and it should be appreciated that modifications and additions may be made thereto without departing from the scope thereof.

1-30. (canceled)

31. A system for providing recommendations for cannabinoid containing medicines to administer to a patient, the system comprising:
an input system for receiving patient information including at least one piece of genetic information,
wherein the at least one piece of genetic information includes information relating to the presence or absence of at least one single nucleotide polymorphism (SNP) associated with a risk factor for administration of a cannabinoid, and
at least one single nucleotide polymorphism (SNP) associated with the patient's likely metabolism rate of at least one cannabinoid, and
a processing system that is configured to provide an output indicative of a medicine recommendation for the patient based on the patient information.

32. The system as claimed in claim 31, wherein the output indicative of a medicine recommendation is a prescription for a cannabinoid containing medicine.

33. The system as claimed in claim 31, wherein the patient information further comprises physiological information which comprises one or more of: weight, age, renal function, hepatic function, medical conditions, or medications currently being taken.

34. The system as claimed in claim 31, wherein the SNP associated with a risk factor for administration of a cannabinoid includes a risk factor for psychosis risk due to administration of at least one of administration of at least one of tetrahydrocannabinol (THC), THCV, THC delta 8, THC delta 9 or varin analogues thereof.

35. The system as claimed in claim 34, wherein the SNP associated with psychosis risk is present in the patient's AKT1 gene.

36. The system as claimed in claim 31, wherein the SNP associated with a risk factor for administration of a cannabinoid includes a risk factor for neurocognitive impairment due to administration of at least one of tetrahydrocannabinol (THC), THCV, THC delta 8, THC delta 9 or varin analogues thereof.

37. The system as claimed in claim 36, wherein the SNP associated with neurocognitive impairment is present in the patient's catechol-O-methyltransferase (COMT) gene.

38. The system as claimed in claim 31, wherein the SNP associated with the patient's likely metabolism rate of at least one cannabinoid is associated with the metabolism of one or more of CBD, THC, CBDV, THCV, THC delta 8, THC delta 9 or varin analogues thereof.

39. The system as claimed in claim 38, wherein the SNP associated with the patient's likely metabolism of at least one cannabinoid is present in at least one of the patient's CYP2C9 and CYP2C19 genes.

40. The system as claimed in claim 34, wherein the risk factor for psychosis risk comprises low, medium and high risk indications, and wherein a high psychosis risk results in a medicine recommendation comprising a cannabinoid composition containing substantially zero THC.

41. The system as claimed in claim 40, wherein a medium psychosis risk results in a medicine recommendation comprising a cannabinoid composition containing a THC dosage of less than 1 mg of THC per day.

42. The system as claimed in claim 36, wherein the risk factor neurocognitive impairment comprises low, medium and high risk indications, and wherein a low, medium or high neurocognitive impairment risk results in a medicine recommendation comprising a cannabinoid composition containing a non-zero daily dose of tetrahydrocannabinol (THC) which is determined to have a low-risk of causing neurocognitive impairment.

43. The system as claimed in claim 38, wherein the likely metabolism rate of THC, THCV, THC delta 8, THC delta 9 or varin analogues thereof comprises low, medium and high metabolism indications and the medicine recommendation comprises a cannabinoid composition comprising corresponding low, medium and high concentrations of THC, THCV, THC delta 8, THC delta 9 or varin analogues.

44. The system as claimed in claim 38, wherein the likely metabolism rate of CBD or CBDV comprises very low, low, medium, high and very high metabolism indications and the medicine recommendation comprises a cannabinoid composition comprising corresponding very low, low, medium, high and very high concentrations of CBD or CBDV.

45. The system as claimed in claim 31, wherein the medicine recommendation includes a cannabinoid composition comprising a first active ingredient and a second active ingredient wherein the first active ingredient is CBD or CBDV and the second active ingredient is THC, THCV, THC delta 8, THC delta 9 or varin analogues thereof.

46. The system as claimed in claim 45, wherein the ratio of the first active ingredient to second active ingredient is selected based on the risk factor for administration of a cannabinoid, and/or the patient's likely metabolism rate of at least one cannabinoid.

47. The system as claimed in claim 31, wherein the medicine recommendation comprises a list of formulations, the list of formulations including a first medicine containing a first active compound and a second medicine containing a second active compound.

48. The system as claimed in claim 47, wherein the formulations of first medicine include a first formulation having a first concentration of the first active compound and a second formulation having a second concentration of the first active compound.

49. The system as claimed in claim 47, wherein the formulations of the second medicine include a third formulation having a first concentration of the second active compound and a fourth formulation having a second concentration of the second active compound.

50. The system as claimed in claim 47, wherein the medicine recommendation comprises at least one of the first and second formulations and at least one of the third and fourth formulations.
